(19) United States
(12) Patent Application Publication
(10) Pub. No.: US 2023/0147744 A1
(43) Pub. Date: May 11, 2023

(54) METHODS TO ENRICH ENTEROENDOCRINE CELLS AND THEIR SUBTYPES IN THE CONTIGUOUS, INTESTINAL MONOLAYER SYSTEMS

(71) Applicants: The University of North Carolina at Chapel Hill, Chapel Hill, NC (US); University of Washington, Seattle, WA (US)

(72) Inventors: Nancy L. Allbritton, Seattle, WA (US); Yuli Wang, Lynnwood, WA (US); Christopher E. Sims, Seattle, WA (US); Cecilia Villegas Novoa, Seattle, WA (US)

(21) Appl. No.: 17/916,683

(22) PCT Filed: Apr. 5, 2021
(86) PCT No.: PCT/US2021/025741

§ 371 Date: Oct. 3, 2022

Related U.S. Application Data
(60) Provisional application No. 63/004,537, filed on Apr. 3, 2020.

(51) Int. Cl. C12N 5/071; C12M 1/12

(52) U.S. Cl. CPC C12N 5/0679; C12M 25/04; C12N 2501/30; C12N 2503/02; C12N 2501/415

(57) ABSTRACT

Provided are new strategies, methods and systems, described herein as vasoactive intestinal peptide (VIP)-assisted air-liquid-interface (ALI) culture, to significantly increase the number of enteroendocrine (EEC) and enterochromaffin (EC) cells over the traditional submerged culture, while at the same time maintaining a high barrier integrity of monolayers. The new strategies, methods and systems overcome the limitations of the existing EEC enrichment methods by maintaining high cell viability and barrier integrity and without requiring complicated procedures of cocultures or genetic engineering/induction. The created EEC-enriched, contiguous monolayer platform acts as a robust analytical tool to enable functional studies of hormone secretion from EEC cells with high signal background ratio and repeatability.

### CROSS-REFERENCE TO RELATED APPLICATIONS

This application claims priority to U.S. Provisional Patent Application Ser. No. 63/004,537, filed Apr. 3, 2020, herein incorporated by reference in its entirety.

### STATEMENT OF GOVERNMENT SUPPORT

This invention was made with government support under grant number DK109559 and DK121580 awarded by National Institutes of Health. The government has certain rights to this invention.

### TECHNICAL FIELD

The presently disclosed subject matter is directed to methods to enrich enteroendocrine cells and their subtypes in the contiguous, intestinal monolayer systems.

### BACKGROUND

Enteroendocrine (EEC) cells constitute less than about 1% of the human intestinal epithelial population, nevertheless they produce and secrete more than 20 different hormones that control a variety of critical physiological functions (Table 1).¹ EEC cells comprise at least 15 different subtypes (e.g. enterochromaffin cells, L cells, K cells, M cells, etc.), and among them enterochromaffin (EC) cells are the most abundant one and they comprise about 40% of EEC cells throughout the entire gastrointestinal tract.^{2, 3} EC cells can sense various stimuli from luminal content (e.g. nutrients, microbial metabolites, irritants, toxins, infection, and mechanical stimuli), and synthesize and secrete a biogenic amine serotonin or 5-hydroxy tryptamine (5HT).^{4,5} More than 90% of the human body's total 5HT is located in the EC cells in the intestinal tract, where it is used to regulate gastrointestinal motility, secretion of digestive enzymes and other important functions.⁵

TABLE 1
Intestinal enteroendocrine cell types and secreted hormones

| EEC Cell Type | Secreted Hormones | Known Effects |
|---|---|---|
| A | Ghrelin, nesfatin-1 | Stimulates appetite |
| Enterochromaffin | Serotonin (5HT) | Stimulates appetite and |
| (EC) | | intestinal mobility |
| Enterochromaffin-like | Histamine | Alters stomach acidity |
| (ECL) | | |
| D | Somatostatin | Stimulates colonic |
| | | peristalsis |
| G | Gastrin | Stimulates gastric acid |
| | | secretion |
| I | Cholecystokinin | Stimulates appetite and |
| | (CCK) | intestinal mobility |
| K | Gastric inhibitory | Stimulates insulin secretion |
| | peptide (GIP) | |
| L | GLP-1; GLP-2; | Suppresses appetite and |
| | oxyntomodulin; | inhibits intestinal motility |
| | peptide YY | |
| M | Motilin | Stimulates GI tract motility |
| N | Neurotensin | Stimulates GI tract motility |
| P | Leptin | Suppresses appetite |
| S | Secretin | Regulates GI tract |
| | | secretions |

Although EEC cells play a crucial role in human physiology, their scarcity has posed a hurdle to the in-depth study of their sensory function and hormone secretion. To overcome this challenge, live EEC cells have been isolated from intestine specimens, enriched by fluorescent activated cell sorting (FACS), and maintained in short-term culture for in vitro hormone secretion and signaling studies.^{6, 7} The main bottleneck of this approach is the requirement of fresh intestine specimens in each experiment. In the past decade, in vitro culture techniques of the intestinal epithelial stem cells (IESCs) has opened the door to create primary cell-derived, physiologically relevant, in vitro models of intestines.⁸⁻¹⁰ These systems support the proliferation of IESCs by culturing them within (as organoids) or on the top of (as monolayers) an extracellular matrix (ECM) in the presence of specific growth factors, thus provide an unlimited supply of cells for in vitro studies. Monolayers of differentiated intestinal epithelial cells were established by plating IESCs or IESCs-containing tissues (e.g. dissociated organoids) directly on the porous membrane to grow to confluence followed by spontaneous or forced differentiation.¹¹⁻¹⁵ The differentiated monolayers displayed characteristic polarized morphology and immunofluorescence markers of intestinal epithelium such as brush border proteins and tight junctions, and contained a diversity of intestinal epithelial cell lineages. EEC cells were identified in these in vitro systems (organoids and monolayers) but their presence is still as rare as in the intestine.^{11, 12}

Several strategies have been developed to increase the presence of EEC and EC cells in the in vitro systems. Coculturing epithelial monolayer system with neurons and myofibroblasts was found to promote differentiation into EEC cells and increased their fraction from 0.3% to 0.9%.¹⁶ In an organoid system, forced differentiation with combined EGFR/Wnt/MAPK inhibitors produces EEC cells with high fraction (about 50%).¹⁷ However, this method generated a high cell death. When this protocol was applied to the monolayer system with 100% surface coverage, only <10% surface remained with live cells after 4-day treatment EGFR/Wnt/MAPK inhibitors (unpublished data). Recently, lentivirus transduction was used to stably engineer IESCs with doxycycline-inducible expression of neurogenin-3 (NGN3), a transcription factor that drives EEC differentiation.³ EEC fraction increased from 0.4% (noninduced) to 40% (induced with doxycycline). Although this model showed some promise, the method modifies the cell genome, and the effect of genetic modification on the cell behaviors needs to be elucidated. Thus, this approach is also not ideal.

The above strategies suffer from several disadvantages such as either low cell viability, low barrier integrity, and/or complicated procedures. What is needed is to establish a simple protocol to enrich EEC cells with only minor modifications of the current established monolayer protocol. The new protocol should overcome the limitations of the above strategies by maintaining high cell viability and barrier integrity and without requiring complicated procedures of cocultures or genetic engineering/induction. Such needs are met, and obstacles overcome, by the instant disclosure.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Provided in some embodiments are methods of producing a live cell construct comprising enteroendocrine cells and subtypes of enteroendocrine cells. Such methods can in some aspects comprise culturing stem cells that are capable of differentiating into enteroendocrine (EEC) cells on an upper surface of a cell support structure, the cell support structure having both an upper surface and a lower surface, until at least a portion of the upper surface of the cell support structure is substantially covered by the stem cells, and causing the stem cells to differentiate into EEC cells by maintaining a thin layer of fluid at the upper surface of the support structure, wherein the stem cells generate a live cell construct comprising a substantially continuous cell monolayer comprising EEC cells and subtypes of EEC cells.

In some embodiments, such methods can further comprise adding one or more compounds in an expansion medium to prevent early lineage-fate decision of stem cells during a proliferation stage, optionally wherein the formation of EEC cells can be further enhanced by addition of the one or more compounds in the expansion medium. The one or more compounds are selected from a Wnt signaling activator and/or a Wnt signaling enhancer, optionally wherein the signaling activator comprises CHIR99021, WAY316606, ABC99, IQ1, and/or arylpyrimidine, and the Wnt signaling enhancer comprises proteins of Wnt-3A and/or R-spondin.

In some embodiments, for the methods, systems, constructs and devices herein, the thin layer of fluid comprises a liquid, slurry, hydrogel, and/or semi-solid materials. The thin layer of fluid at the upper surface can be maintained in a range of about 0.001 mm to about 10 mm, optionally about 0.001 mm to about 1 mm, above the luminal side of the cell monolayer. The thin layer of fluid at the upper surface can be maintained by adding hormones and/or compounds to the medium composition to induce luminal fluid liquid secretion, optionally wherein the hormones and/or compounds comprise chemicals (e.g. ionomycin), hormones (e.g. vasoactive intestinal peptide, serotonin, gastrin, etc.), cytokines, metabolites, bacteria and/or bacteria components, optionally further adding hypertonic medium to the luminal side to stimulate fluid secretion towards the luminal side to create a thick mucus layer. The thin layer of fluid can be maintained by a microfluidic flow setup. High barrier integrity can also be maintained in some embodiments.

In some embodiments, for the methods, systems, constructs and devices herein, the EEC cells can secrete serotonin. The EEC cells can secrete glucagon-like peptide-1 (GLP-1). The EEC cells secrete peptide YY (PYY), or other intestinal hormones including cholecystokinin, motilin, neurotensin, leptin and/or secretin. The EEC cells can also comprise L-cells, wherein a density of the L-cells is greater than about 50 cells/mm², optionally greater than about 100 cells/mm².

In some embodiments, the methods disclosed herein can be used for screening of drug, metabolite, foodstuff or compound-induced secretion of hormones from EEC cells. The methods can be used for screening of drug, metabolite, foodstuff or compounds that block secretion of hormones from EEC cells. The methods can be used for screening of drug, metabolite, foodstuff or compounds that potentiate secretion of hormones from EEC cells. The gastrointestinal epithelial cells can be selected from the group consisting of mammalian, avian, reptilian, amphibian, and insect cells. The gastrointestinal epithelial cells can be human gastrointestinal epithelial cells.

In some embodiments, for the methods, systems, constructs and devices herein, the gastrointestinal epithelial cells can be selected from the group consisting of colon, small intestine, stomach, esophagus, tongue, nasopharynx, oropharynx, laryngeopharynx, and pancreatic epithelial cells.

Provided herein are live cell constructs produced by the method of any of the above claims, wherein the live cell construct comprises a substantially continuous cell monolayer comprising EEC cells and subtypes of EEC cells. The EEC cells in such constructs can secrete serotonin. The EEC cells in such constructs can secrete glucagon-like peptide-1 (GLP-1). The EEC cells in such constructs can secrete peptide YY (PYY), or other intestinal hormones including cholecystokinin, motilin, neurotensin, leptin and/or secretin. Such constructs can comprise L-cells, optionally wherein a density of the L-cells is greater than about 50 cells/mm², optionally greater than about 100 cells/mm².

In some embodiments, provided herein are live cell culture systems, the systems comprising a cell support structure, the cell support structure having both an upper surface and a lower surface, the cell support structure further comprising a porous carrier on the upper surface, a culture vessel housing the cell support structure and providing a contained area for a culture medium, wherein the culture medium is contained below the lower surface of the cell support structure, wherein the cell support structure is configured to generate a live cell construct from stem cells seeded on the cell support structure, the system configured to cause the stem cells to differentiate into enteroendocrine (EEC) cells by maintaining a thin layer of fluid at the upper surface of the support structure, wherein the thin layer of fluid at the upper surface is maintained by hormones and/or compounds in the culture medium contained in the culture vessel inducing luminal fluid liquid secretion. Such systems can be configured to generate a substantially continuous cell monolayer comprising EEC cells and subtypes of EEC cells. In some embodiments, the thin layer of fluid in such systems can be maintained by a microfluidic flow of the cell support structure. The culture vessel can comprise a multi-well plate, culture dish, vial or tube. The thin layer of fluid at the upper surface can be maintained by hormones and/or compounds in the culture medium that induces luminal fluid liquid secretion, optionally wherein the hormones and/or compounds comprise chemicals (e.g. ionomycin), hormones (e.g. vasoactive intestinal peptide, serotonin, gastrin, etc.), cytokines, metabolites, bacteria and/or bacteria components, optionally further adding hypertonic medium to the luminal side to stimulate fluid secretion towards the luminal side to create a thick mucus layer.

In some embodiments, provided herein are methods of screening a test compound or microbe for a toxicological, physiological, or carcinogenic effect, comprising: (a) providing a cell construct according to any of the above claims; (b) contacting a test compound or microbe to the cell construct; and then (c) detecting a toxicological, pharmacologic physiological, or carcinogenic effect of the microbe on cells of the cell construct, optionally by comparing the cell construct after the contacting to a like cell construct to which the compound or microbe has not been contacted, and/or by comparing the cell construct after contacting with the cell construct before the contacting step. The test compound or microbe can be selected from the group consisting of aromatic organic compounds, aliphatic organic compounds, and mixed aromatic and aliphatic organic compounds. The test compound or microbe can be selected from the group consisting of gram negative bacteria, gram positive bacteria, yeast, and molds. Such methods can comprise screening for pharmacologic interventions for diabetes. Such methods can comprise screening for pharmacologic interventions for obesity.

Accordingly, these and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A-1C include schematics showing the difference among three cell culture setups. FIG. 1A shows a submerged culture where the apical surface (or luminal side) is covered with medium. FIG. 1B shows an air-liquid-interface (ALI) culture where the apical surface is exposed to air and completely dry. FIG. 1C shows a vasoactive intestinal peptide (VIP)-assisted ALI culture, where VIP stimulates the cells to secrete and maintain a thin layer of water at the apical side. This thin layer of liquid hydrates the apical surface.

FIGS. 2A-2C include experimental results showing that the scarcity of EEC and EC cells in the in vitro monolayer platform reduce the reliability of 5HT secretion assay. FIG. 2A is fluorescence microscopic images of monolayers generated by traditional submerged culture. Left is the stitched image showing the entire surface of 24-well inserts (surface area=33 mm²). Middle and right are close ups. Markers are as follows: chromogranin A (ChgA, EEC marker), red; 5HT (EC marker); Hoechst 33342-stained nuclei. FIG. 2B shows the quantification of the number of EECs (ChgA+) and ECs (5HT+) on monolayers. FIG. 2C shows the 5-HT secretion from monolayers after apical exposure to DMSO vehicle and 10 μM forskolin for 4 h. Three independent experiments were performed. Sample number=3 for each condition. A t-test was used to perform statistical analysis. *: p<0.05, NS: not significant.

FIGS. 3A and 3B illustrate that the number of EEC and EC cells on human stem-cell-derived intestinal epithelial monolayers is dependent on the differentiation strategies. FIG. 3A is a schematic showing methods or processes to generate fully differentiated, confluent monolayers derived from primary intestinal epithelial stem cells by four differentiation strategies: submerged (Sub) and air-liquid-interface (ALI) in the absence or presence of VIP. FIG. 3B is representative low-magnification fluorescence microscopic images of monolayers. Inserts show high-magnification images. Markers as follows: chromogranin A (ChgA, EEC marker); 5HT (EC marker); Hoechst 33342-stained nuclei. The last strategy, VIP-assisted ALI culture, generated the monolayers with the most abundant EEC and EC cells.

FIGS. 4A-4F show results from the characterization of the EC-enriched monolayers. FIG. 4A shows the quantification of the number of EEC (ChgA+) cells on monolayers generated by four differentiation strategies. FIG. 4B shows the quantification of the number of EC (5HT+) cells. FIG. 4C shows the 5HT secretion from monolayers after apical exposure to DMSO vehicle and 10 μM forskolin for 4 h. FIG. 4D shows box plot of apparent permeability coefficient (Pₐₚₚ) of monolayers showing the monolayer integrity is best for samples generated from VIP-assisted ALI culture. Pₐₚₚ of 4×10⁷ cm/s is a commonly accepted threshold for barrier integrity. FIGS. 4E-4F shows the effect of differentiation duration (3-7 days) on 5HT secretion (FIG. 4E) and barrier integrity (FIG. 4F, box plot). In FIGS. 4D and 4F, sample number=6 for each condition. In other figures, sample number=3 for each condition. A t-test was used to perform statistical analysis: *p<0.05, **p<0.005.

FIG. 5A shows 5HT secretion from monolayers generated from stem cells at different passage number. FIG. 5B shows a list of information (age and sex) of stem cells from five donors. FIG. 5C shows 5HT secretion from monolayers generated from transverse colonic stem cells from five donors. In other figures, sample number=3 for each condition. A t-test was used to perform statistical analysis: *p<0.05, **p<0.005.

FIGS. 6A-6D show results of a small scale compound screen for modulation of 5HT secretion. FIG. 6A is confocal fluorescence microscopy images (XY and XZ) showing triangular or pyramidal EC in shape. EC cells exhibit yellow color due to co-stain with 5HT (green; see arrow label) and ChgA (red; see arrow label). Nuclei are stained with Hoechst 33342 (blue; see arrow label). FIG. 6B is a schematic of EC showing that dietary compound can stimulate the apically located receptors followed by the release of 5HT through basolateral boundary. FIG. 6C is a list of 13 dietary pungent ingredients, plant source and work concentration. FIG. 6D shows 5HT secretion from monolayers after apically stimulated with dietary compounds for 4 h. Sample number=3 for each condition. A t-test was used to perform statistical analysis over vehicle control: *p<0.05, **p<0.005.

FIG. 7 confirms the presence of L-cells in the monolayers. In the left panel the monolayer was stained with GLP-1 (green, Santa Cruz, sc-514592, 1:200) and Hoechst 33342 (blue). In the right panel the monolayer was stained with PYY (red, Abcam, ab22663, 1:200) and Hoechst 33342 (blue).

FIGS. 8A-8B show the optimization of differentiation medium and culture format to enrich L-cells. FIG. 8A shows fluorescence microscopic images of monolayers. GLP-1: green. Nuclei: blue. FIG. 8B is the quantification of GPL-1⁺ L cells. Sample number=3. * p<0.05. ** p<0.005. #N.S.

FIGS. 9A-9C show the optimization of differentiation time. FIGS. 9A and 9B show the quantification of L-cells by GLP-1 (FIG. 9A) and PYY (FIG. 9B) markers. FIG. 9C shows the fluorescence microscopic images of monolayers imaged at XY and YZ planes. Sample number=3. * p<0.05. ** p<0.005. # N.S.

FIG. 10 shows the secretion of GLP-1 to the basal compartment by 4-h apical stimulation of a variety of compounds. Concentration of stimuli: Glucose=10 mM, forskolin=10 μM, TGR5=10 μM, IBMX=10 μM, KCl=30 mM, Bombesin=10 μM. Sample number=3. * p<0.05. ** p<0.005. # N.S compared to control.

FIGS. 11A-11B show the test Wnt activator (CHIR99021) in EM during the proliferation stage. FIG. 11A shows fluorescence images of monolayers showing the abundance of EC (5HT⁺) cells is increased when the stem cells were cultured in the presence of CHIR prior to being differentiated in conventional submerged culture. FIG. 11B is the quantification of EC cell density. Sample number=3 for each condition. A t-test was used to perform statistical analysis over vehicle control: *p<0.05, **p<0.005.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

Provided herein in some embodiments is a simple strategy, VIP-assisted ALI culture, to significantly boost the number of EEC and EC cells over the traditional submerged culture, while at the same time maintain a high barrier integrity of monolayers. This new strategy overcomes the limitations of the existing EEC enrichment methods by maintaining high cell viability and barrier integrity and without requiring complicated procedures of cocultures or genetic engineering/induction. The created EEC-enriched, contiguous monolayer platform acts as a robust analytical tool to enable functional studies of hormone secretion from EEC cells with high signal background ratio and repeatability.

General Considerations

Cells such as undifferentiated cells and/or gastrointestinal epithelial cells used to carry out the present invention may be of any species of origin, including for example, but not limited to, mammalian, avian, reptile, amphibian, and insect. In some embodiments the cells are mammalian cells, examples of which include but are not limited to, human, monkey, ape, goat, sheep, dog, cat, horse, cow, and pig gastrointestinal epithelial cells. In some embodiments, the cells are preferably derived from primary tissues, and are not cancer or tumor cells. Any type of gastrointestinal epithelial cells may be used, including, but not limited to, colon, small intestine, stomach, esophagus, tongue, nasopharnyx, oropharynx, laryngeopharynx, and pancreatic epithelial cells.

Chemical screens of many compounds, factors, metabolites, foodstuffs, small molecules and/or drugs can be used to regulate the cell signaling pathways to induce the EEC cell formation as well as to block or enhance their secretion. The factors, small molecules and drugs include activators and inhibitors of Wnt, BMP, GREM1,2, Notch signaling pathways. Non-limiting examples are CHIR99021 (Wnt activator), IWP (Wnt inhibitor), Y-27632 (Notch inhibitor), Noggin (BMP inhibitor), Jagged 1 (Notch activator), Gremlin (BMP antagonist), cytokines, dietary compounds (fiber, butyrate, other fatty acids, metabolites), etc. Other fatty acids include propionate and acetate, which are short-chain fatty acids produced by microbial fermentation of fiber, and additional metabolites include branched chain fatty acids, bile acids and microbial-derived secondary bile acids, urea, amines, ammonia, lactate, phenols, indoles, sulfurs, carbon dioxide, hydrogen, hydrogen sulfide, and methane. Metabolites include those from complex carbohydrates (soluble fiber), beans, and resistant starches, and can be produced from microbiota. Other chemicals include antidiuretic hormone, laxatives, bacterial endotoxins, hormones (e.g., VIP), and endogenous substances (e.g., bile acids), aldosterone, somatostatin, alpha2-adrenergic agents (e.g., clonidine), acetylcholine, nitric oxide, adenosine triphosphate (ATP), etc.

### APPLICATIONS AND USES OF THE PRESENT DISCLOSURE

The proposed EEC cell enriched surface may be planar or convoluted but is characterized by having an open architecture unlike the organoids which are closed structures. By providing a noncancerous culture system characterized by an open architecture, the present disclosure has overcome the limitations of the organoid system making the culture of EEC-enriched tissues composed of primary cells compatible with conventional tissue culture methods and current robotics used in automated, high-throughput culture and analysis platforms. The open architecture and permeable substrate make possible culture of cells with application of different maturation agents to either cell surface (basal/apical or luminal) as well as measurement of secretion from either surface. The open architecture with enriched EEC cells will enable assays secretion along with or without high epithelial barrier function with high sensitivity. Interactions of the EEC cells with overlying bacteria and other components of a microbiome are also now possible. These ex vivo tissues with enriched EEC cells can be created from a variety of species including mouse, pig, and human among others. The ability to create these tissues from healthy and diseased sources and from cells of differing genetic backgrounds will be important for screening drugs, study of disease mechanisms, and study of basic biology of EEC cells. Addition of various other cell types (e.g. immune cells, fibroblasts, and others found co-existing with the particular epithelial tissue in vivo) co-cultured on or within the biomimetic scaffold will be valuable for understanding EEC cell-cell interactions and the effect of drugs and metabolites on the EEC cells. These EEC-enriched epithelial tissues are superior to the current EEC cell models based on an understanding the physiology, toxicology and pharmacology of EEC cells. Some examples follow but this list is not all inclusive.
1) Screening studies of drugs, biologics, toxins, mutagens, dietary compounds, pathogens, viruses, microbiota, etc.
2) Disease models by using primary cells derived from a translational animal models or human
3) Pharmacological and pharmacokinetic models for screening including comprehensive dose-response profiles for drugs, dietary compounds, etc.
4) In vitro models for study EEC cell-neuronal interactions
5) Personalized medicine by studies performed on specific genetic backgrounds and individual patients
6) Identification of compounds in screening for therapeutics for metabolic diseases, obesity, and diabetes
7) Assays of syn-, pre- and probiotic agents on EEC cell function.
8) Impact of immune cells and their products (antibodies and cytokines) on EEC cells

Screening Methods

Thus, as noted above, in some embodiments the present disclosure provides a method of screening a test compound or microbe for a toxicological, physiological, or pharmacologic effect exerted by or on EEC cells: (a) providing a cell construct as described herein; (b) contacting a test compound or microbe to said construct; and then (c) detecting a toxicological, physiological, or carcinogenic effect of said microbe on the cells of the cell construct (e.g., by comparing the cell construct after the contacting to a like cell construct to which said compound or microbe has not been contacted, and/or by comparing the construct after the contacting step to the cell construct before said contacting step).

In some embodiments, the test compound or microbe is selected from the group consisting of aromatic organic compounds, aliphatic organic compounds, and mixed aromatic and aliphatic organic compounds. For example, in some embodiments, the compounds for screening are compounds are natural products, prebiotics, probiotics, foodstuffs, food metabolites, carcinogens, drugs, drug metabolites, bacterial metabolites and toxins, irritants, soil compounds, ingestible toxins, etc.

In some embodiments, the test compound or microbe is selected from the group consisting of gram negative bacteria, gram positive bacteria, yeast, and molds. For example, in some embodiments, the microbe is a bacteria of a type found in the ordinary or healthy gut flora (or “microbiome”) of mammalian, particularly human, species. See, e.g., US Patent Application Publication No. US 2014/0093478. In some embodiments, the microbe is an infectious organism, such as Clostridium, cholera, Salmonella, Shigella, worms (tape, pin, hook, eyc), amoeba (giardia, etc), etc. Thus, in some embodiments, the microbe is an enteric bacteria or pathogen, including both benign and infectious enteric bacteria and pathogens.

Suitable detection methods include, but are not limited to, ELISA, electrochemistry, immunohistochemistry, PCR for DNA, mRNA expression, RNA sequencing, transepithelial electrical resistance, transport assays (ion, compound, protein, etc.), secretion assays, electron microscopy, flow cytometry, mass spectrometry of supernatants or reservoirs, and radiochemistry assays of the same, fluorescence based sensors of the same, and microbe adhesion to the epithelial cells.

While particular examples of colonic monolayers are given, it will be appreciated that monolayers from other gastrointestinal epithelial cells can also be formed, particularly small intestine, intestine, stomach, esophagus, tongue, nasopharynx, oropharynx, laryngeopharynx, and pancreatic epithelial cells, etc., in like manner as described below or by variations of such techniques that will be apparent to those skilled in the art.

General Discussion of VIP-Assisted ALI Culture

An air-liquid interface (ALI) culture can be prepared in which liquid or medium is removed from the apical reservoir, or luminal side, or luminal reservoir, all used interchangeably. Being different from traditional submerged culture, the apical surface of monolayers in ALI culture is exposed to air and completely dry. This situation does not reflect the in vivo intestinal luminal environment due to the absence of a high water content at the apical surface. Indeed, water and electrolyte homeostasis of the colonic mucosa are balanced with water moving into and out of the lumen. To improve the ALI culture, this present disclosure used an intestinal hormone, vasoactive intestinal peptide (VIP), to assist in the balance of fluid movement across the epithelium so that a thin layer of fluid (about 0.1 mm to about 1.0 mm in thickness, or about 0.3 mm to about 0.5 mm in thickness, optionally about 0.4 mm in thickness) was maintained at the apical reservoir to prevent the dry up. This combination of VIP and ALI culture, referred to herein as “VIP-assisted ALI culture”, significantly increased the number of EEC cells compared with ALI or submerged culture alone. An additional advantage of VIP-assisted ALI culture is the improved barrier integrity (see Example 2 for details). Without being bound by any particular theory or mechanism of action, in some embodiments VIP-assisted ALI culture can maintain a thin layer of fluid (about 0.1 mm to about 1.0 mm in thickness, or about 0.3 mm to about 0.5 mm in thickness, optionally about 0.4 mm in thickness) at the apical side, thus increasing the oxygen availability and at the same time eliminating the stress from drying up. The present disclosure overcomes the limitations of traditional ALI or submerged cultures.

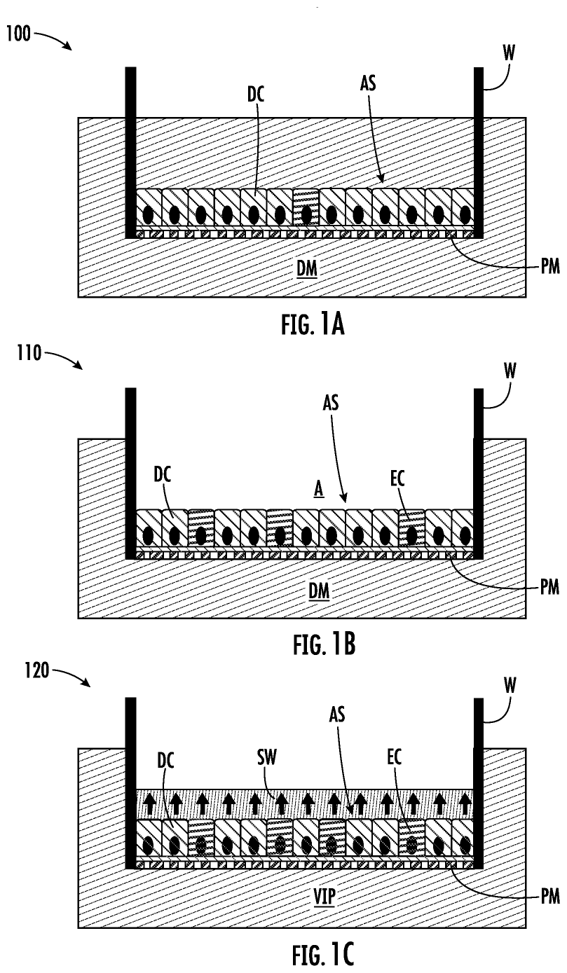

Referring now to the figures, FIGS. 1A-1C include schematics showing the difference among three cell culture setups. FIG. 1A is a cross-sectional schematic representation of a submerged culture 100 where the apical surface AS (or luminal side of the culture) of the differentiated cells DC is covered with differentiation medium DM. Conversely, FIG. 1B is a cross-sectional schematic representation of an air-liquid-interface (ALI) culture 110 where the apical surface AS is exposed to air A and completely dry. Finally, FIG. 1C is a cross-sectional schematic representation of a vasoactive intestinal peptide (VIP)-assisted ALI culture 120, where vasoactive intestinal peptide VIP stimulates the cells (differentiated cells DC and enterochromaffin cells EC) to secrete and maintain a thin layer of water, or secreted water SW, at the apical surface AS. This thin layer of liquid, or secreted water SW, hydrates the apical surface AS. In each of FIGS. 1A-1C the cultures can be in a culture well W or other suitable culture apparatus, culture vessel, system or device as known to those of ordinary skill in the art, including but not limited to a multi-well plate, agar plate, culture dish, vial, tube or the like. In each of FIGS. 1A-1C the cultures include a porous membrane PM upon which the cells (differentiated cells DC and enterochromaffin cells EC) are cultured.

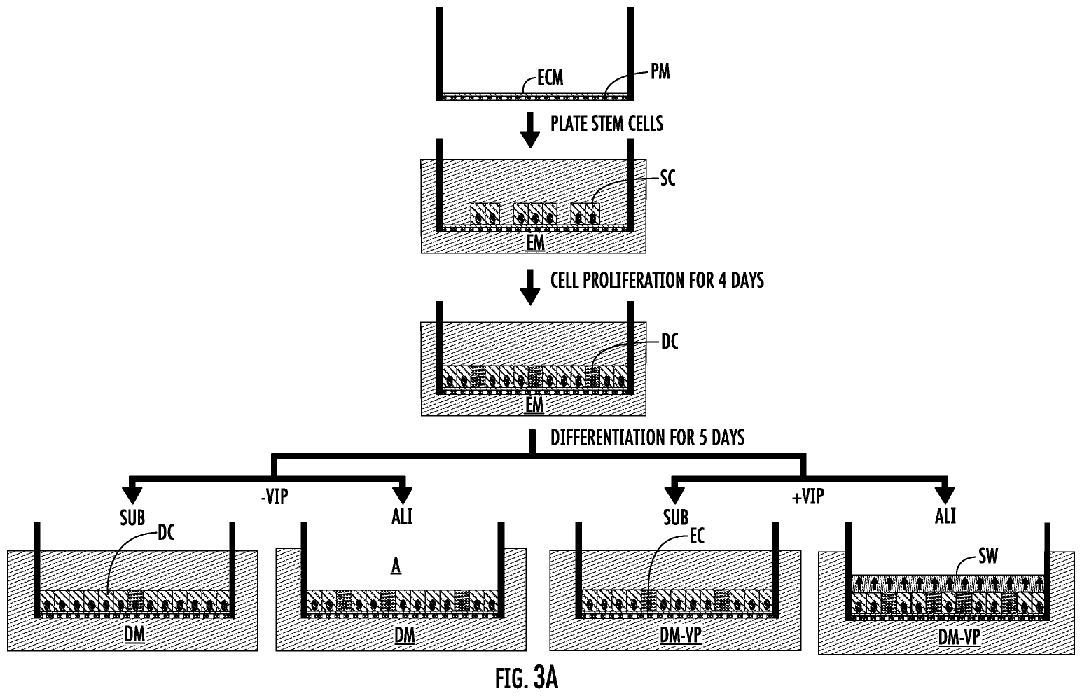

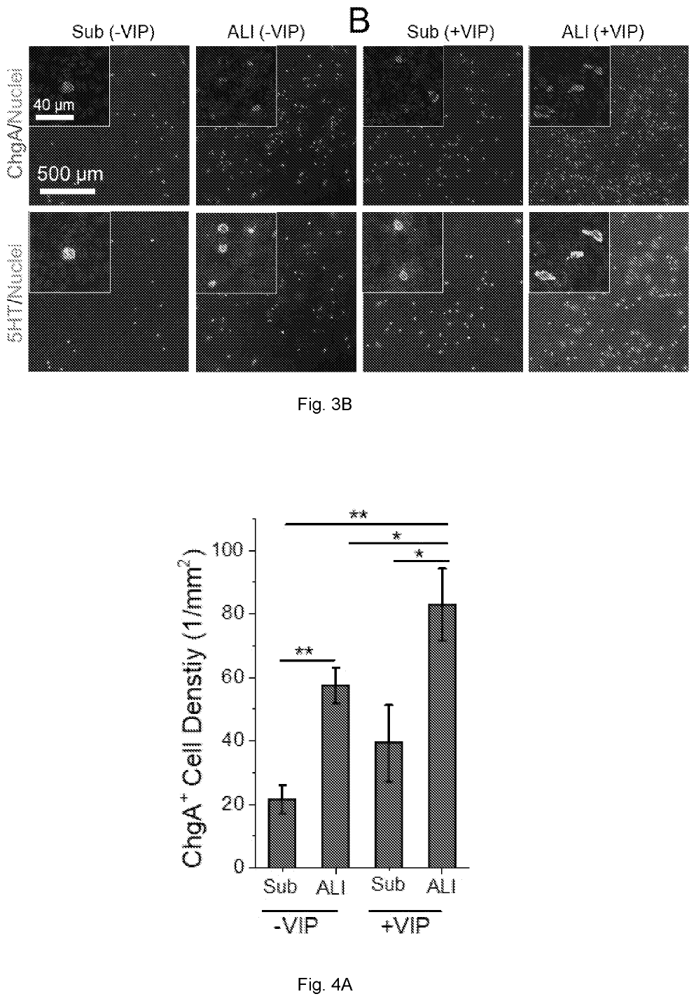

To elaborate further, FIG. 3A illustrates that the number of EEC and EC cells on human stem-cell-derived intestinal epithelial monolayers is dependent on the differentiation strategies. FIG. 3A is a schematic representation of methods or processes to generate fully differentiated, confluent monolayers derived from primary intestinal epithelial stem cells SC by four differentiation strategies: submerged (SUB) and air-liquid-interface (ALI) in the absence or presence of VIP. As shown in FIG. 3A, stem cells SC were plated on extracellular matrix ECM on a porous membrane PM. Cells were proliferated for about four days in a medium EM. The cells were then differentiated for up to five days using one of the four differentiation strategies. The results of the four differentiation strategies are shown in FIG. 3B. FIG. 3B is representative low-magnification fluorescence microscopic images of monolayers. Inserts show high-magnification images. Markers as follows: chromogranin A (ChgA, EEC marker); 5HT (EC marker); Hoechst 33342-stained nuclei. The last strategy, VIP-assisted ALI culture, generated the monolayers with the most abundant EEC and EC cells.

Methods to Enrich Enteroendocrine Cells in Monolayer Systems

In some aspects, provided herein are methods of producing a live cell constructs comprising a cell monolayer comprising enteroendocrine cells and subtypes of enteroendocrine cells. Such methods can comprise culturing stem cells that are capable of differentiating into enteroendocrine (EEC) cells on an upper surface of a cell support structure having both an upper surface and a lower surface until at least a portion of the upper surface of the cell support structure is substantially covered by the stem cells, and differentiating the stem cells by maintaining a thin layer of fluid at the upper surface, wherein the stem cells generate a live cell construct comprising a substantially continuous cell monolayer comprising enteroendocrine cells and subtypes of enteroendocrine cells. The thin layer of fluid can comprise liquid, slurry, hydrogel, and/or semi-solid materials. The thin layer of fluid at the upper surface can be maintained in a range of about 0.001 mm to about 10 mm, optionally about 0.001 mm to about 1 mm, optionally about 0.3 mm to about 0.5 mm in thickness, optionally about 0.4 mm in thickness above the luminal side of the cell monolayer. In some aspects, the thin layer of fluid at the upper surface is maintained by adding hormones and/or compounds to the medium composition to induce luminal fluid liquid secretion, optionally wherein the hormones and/or compounds comprise chemicals (e.g. ionomycin), hormones (e.g. vasoactive intestinal peptide, serotonin, gastrin, etc.), cytokines, metabolites, bacteria and/or bacteria components, optionally further adding hypertonic medium to the luminal side to stimulate fluid secretion towards the luminal side to create a thick mucus layer. Moreover, in some embodiments, the thin layer of fluid can be maintained by a microfluidic flow setup.

In some aspects, such methods allow for a high barrier integrity to be maintained in the cultures. Some EEC (e.g. EC cells) can secrete serotonin. Some EEC cells (such as L cells) can secrete glucagon-like peptide-1 (GLP-1). Some EEC cells can secrete peptide YY (PYY), or other intestinal hormones including cholecystokinin, motilin, neurotensin, leptin and/or secretin.

In some embodiments, such methods are used for screening of drug, metabolite, foodstuff or compound-induced secretion of hormones from EEC cells. These methods can also be used for screening of drug, metabolite, foodstuff or compounds that block secretion of hormones from EEC cells. The gastrointestinal epithelial cells can be selected from the group consisting of mammalian, avian, reptilian, amphibian, and insect cells. In some embodiments, the gastrointestinal epithelial cells are human gastrointestinal epithelial cells. In some aspects, the gastrointestinal epithelial cells are selected from the group consisting of colon, small intestine, stomach, esophagus, tongue, nasopharynx, oropharynx, laryngeopharynx, and pancreatic epithelial cells.

In some embodiments, the methods further comprise adding one or more compounds in an expansion medium to prevent early lineage-fate decision of stem cells during a proliferation stage, optionally wherein the formation of EEC cells can be further enhanced by addition of the one or more compounds in the expansion medium. The one or more compounds can be selected from a Wnt signaling activator and/or a Wnt signaling enhancer, optionally wherein the signaling activator comprises CHIR99021, WAY316606, ABC99, IQ1, and/or arylpyrimidine, and the Wnt signaling enhancer comprises proteins of Wnt-3A, R-spondin1, and other Wnt's e.g. Wnt's 1 through 16 and other Rspondins e.g. Rspondin's 1 through 4.

Live cell constructs produced by the methods disclosed herein are also provided. The live cell constructs can comprise a cell monolayer comprising enteroendocrine cells and their subtypes.

### Definitions

Unless otherwise defined, all technical and scientific terms used herein have the same meaning as commonly understood by one of ordinary skill in the art to which this presently disclosed subject matter belongs. The terminology used in the description of the presently disclosed subject matter herein is for the purpose of describing particular embodiments only and is not intended to be limiting of the presently disclosed subject matter.

All publications, patent applications, patents and other references cited herein are incorporated by reference in their entireties for the teachings relevant to the sentence and/or paragraph in which the reference is presented.

Unless the context indicates otherwise, it is specifically intended that the various features of the presently disclosed subject matter described herein can be used in any combination. Moreover, the presently disclosed subject matter also contemplates that in some embodiments of the presently disclosed subject matter, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Like numbers refer to like elements throughout. In the figures, the thickness of certain lines, layers, components, elements or features can be exaggerated for clarity. Where used, broken lines illustrate optional features or operations unless specified otherwise.

As used in the description of the presently disclosed subject matter and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value can include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

Spatially relative terms, such as “under,” “below,” “lower,” “over,” “upper” and the like, can be used herein for ease of description to describe an element's or feature's relationship to another element(s) or feature(s) as illustrated in the figures.

It will be understood that, although the terms first, second, etc., can be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section. Thus, a first element, component, region, layer or section discussed herein could be termed a second element, component, region, layer or section without departing from the teachings of the presently disclosed subject matter. The sequence of operations (or steps) is not limited to the order presented in the claims or figures unless specifically indicated otherwise.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

In some embodiments, stem cells useful with the present disclosure can include, but are not limited to, epithelial stem cells, intestinal epithelial stem cells, basal stem cells, induced pluripotent stem cells, respiratory stem cells, gastric stem cells, nasal stem cells, reproductive tract cells (cervix, vagina, uterus), urethra cells, olfactory cells, mouth cells, tongue cells, and/or conjunctiva cells. In some embodiments, the stem cells are intestinal epithelial stem cells.

In some embodiments, a live cell construct comprising a cell monolayer can comprise one or more different cell types (e.g., 1, 2, 3, 4, 5, or more). A “cell type” as used herein refers to morphologically or phenotypically distinct cell forms within a species. In some embodiments, the cells positioned on a cell support structure can be from healthy, inflamed, or diseased human or animal tissue. In some embodiments, cells useful for making a live cell construct of the presently disclosed subject matter can be from human or animal tissue having a disease including but not limited to, inflammatory bowel disease, constipation, cystic fibrosis irritable bowel syndrome, leaky gut syndrome, bacterial overgrowth syndromes, celiac disease, lactose intolerance, excessive gas syndromes, diarrheal diseases, and/or polyps appendicitis.

In some embodiments, a cell layer of the presently disclosed subject matter can be flat, 2-dimensional as illustrated, for example, in FIGS. 1A through 1C. In some embodiments, a cell monolayer of the presently disclosed subject matter can also be folded in a 3-dimensional shape or structure to mimic, for example, the crypt structure or crypt-villus structure of in vivo intestines.

In some embodiments, the methods of the present presently disclosed subject matter can further comprise positioning an impermeable physical barrier and/or a partially permeable (i.e., semi-permeable) physical barrier on or over the luminal side of the cell monolayer comprising mucus producing cells (and on or over/above the mucus layer). In some embodiments, water transit can be regulated by controlling liquid/water movement or water vapor movement.

In some embodiments, the volume of the liquid medium in the luminal (apical) reservoir (with or without a physical barrier), or on the luminal side of the cells, can be a depth in a range of about 0.001 mm to about 10 mm above the luminal side of the cell monolayer (e.g., about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 10 mm, or any value or range therein) (e.g., about 0.005 mm to about 10 mm, 0.01 mm to about 10 mm, about 0.05 mm to about 10 mm, 0.1 mm to about 10 mm, about 0.5 mm to about 10 mm, 1 mm to about 10 mm, about 5 mm to about 10 mm, about 0.001 mm to about 1 mm, about 0.005 to about 1 mm, 0.01 mm to about 1 mm, about 0.05 mm to about 1 mm, 0.1 mm to about 1 mm, about 0.5 mm to about 1 mm, about 0.001 mm to about 0.1 mm, about 0.005 to about 0.1 mm, 0.01 mm to about 0.1 mm, about 0.05 mm to about 0.1 mm, 0.1 mm to about 0.1 mm, about 0.5 mm to about 0.1 mm, or any value or range therein).

As used herein, a “partially permeable physical barrier”, or “porous carrier”, is impermeable or substantially impermeable to mucin, but water can pass through the barrier. Thus, in some embodiments, a partially permeable physical barrier can have a molecular weight cut-off (MWCO) of about 100 kDa, i.e., the barrier is impermeable to molecules greater than (>) about 100 kDa. In some embodiments, the partially permeable barrier can have a MWCO of about 100 to about 150 kDa). Mucin has molecular weight of about 200 kDa-200 MDa.

As used herein an “impermeable physical barrier” is at least substantially, and preferably completely, impermeable to the liquid medium (e.g., water) and to mucin.

Thus, in some embodiments, an impermeable physical barrier or a partially permeable physical barrier (or permeable carrier) can be used to confine mucins on or near the surface of a mucin producing cell monolayer. In some embodiments, an impermeable physical barrier and/or a partially permeable physical barrier can be used to prevent or reduce the dilution by the liquid medium of the mucin as it is produced by the cell monolayer.

Non-limiting examples of physical barriers include a semi-liquid mass (e.g. hydrogels), a gas-impermeable membrane, a gas permeable membrane, and hygroscopic materials (honey, glycerin, sugar, nylon, ABS (acrylonitrile/butadiene/styrene), polycarbonate, cellulose, and poly(methyl methacrylate)). In some embodiments, partially permeable (e.g., molecular weight cutoff of about 100 kDa) physical barriers can include but are not limited to porous materials including porous membranes, some synthetic polymers, hydrogels (e.g., agarose, gelatin, collagen, Matrigel®, etc.), some oils, and/or meshes (e.g., nylon, photoresists, polydimethylsiloxane and other synthetic polymers, etc.). In some embodiments, a vapor permeable physical barrier can be used. Nonlimiting examples of vapor permeable membranes useful with the presently disclosed subject matter include polydimethylsiloxane (PDMS) without coatings/fillers, some synthetic polymers, and/or meshes. Non-limiting examples of impermeable membranes (impermeable to water and to mucin) include solid floaters (e.g., waxes, plastics, etc.), meshes (nylon, photoresists, polydimethylsiloxane and other synthetic polymers, etc.), oils (e.g., mineral oils, perfluorocarbons, natural oils etc.), and/or synthetic polymers.

As used herein, a “cell support structure” can be any structure upon which the one or more cells and/or tissue can be positioned and can be organic, inorganic, or a composite thereof including, for example, any porous or mesh membrane.

In some embodiments, a cell support structure can comprise an organic polymer such as collagen, typically in combination with other ingredients as discussed below. In some embodiments, the supports are porous. A support can be provided or mounted on a porous carrier (e.g., a porous membrane, a mesh, an inorganic grid, a hydrogel, or a combination thereof) to lend structural support thereto, as also discussed below. A support can be in any suitable shape or configuration, including flat, tubular, curved, spherical, ellipsoid, etc., including composites there (e.g., to emulate macroanatomical structures).

Thus, a cell support structure useful with the presently disclosed subject matter can include, but is not limited to, a membrane, ECM (extracellular matrix), hydrogel, natural or synthetic polymers, and/or a two- or three-dimensional scaffold and/or any combination thereof. In some embodiments, for example, the bottom wall of a luminal reservoir can be a cell support structure (e.g., a membrane). In some embodiments, a cell support structure can comprise microstructures (e.g., features having a size of less than about 1 mm (e.g., about 100, 200 or 300 microns deep, up to 800 or 1000 microns deep or more, and/or from about 10 or 50 microns wide, up to 100 or 200 microns wide or more; e.g., a microwell, a post, and/or a groove). In some embodiments, a cell support structure can be comprised of, for example, polytetrafluoroethylene (PTFE), polyethylene terephthalate (PET), polycarbonate (PC), polyvinylidiene fluoride (PVDF), polyethersulfone (PES), cellulose acetate, regenerated cellulose, cellulose nitride, nylon, carbon grid, graphene films, glass, Bioglass (e.g., 45S5 Bioglass), hydroxyapatite, calcium phosphate, silicon, silicon oxide, silicon nitride, titanium oxide, aluminum oxide, gold, nickel, and/or stainless steel, or any combination thereof.

In some embodiments, a material useful as a cell support structure of the presently disclosed subject matter that is not naturally porous, can be made porous by methods that include, but are not limited to, sintering, etching, leaching, lithography, laser micromachining, etc. For example, a porous mesh of silicon and gold can be fabricated by lithography/etching. In some embodiments, photoreactive polymers such as photoresist that are fabricated into a film with micro or nanopores or micro or nanomesh by photolithography can be used for a cell support structure. In some embodiments, elastomeric films such as polydimethylsiloxane (PDMS) or EcoFlex that are fabricated into porous film or micro/nanomesh by soft lithography or molding can also be used as cell support structure. In some embodiments, a cell support structure can also be a dehydrated or flexible yet strong matrix such as a collagen or fibrin film or a composite.

Cells and/or tissues can be placed on a cell support structure or scaffold with or without additional adhesion proteins or extracellular matrices. In some embodiments, a scaffold can comprise extracellular matrix (ECM) materials including, but not limited to, collagen, gelatin, laminin, elastin, fibronectin, vitronectin, heparin sulfate, chondroitin sulfate, keratin sulfate, hyaluronic acid, gelatinous protein mixture secreted by Engelbreth-Holm-Swarm mouse sarcoma cells (e.g. Matrigel®, Geltrex®, MaxGel™, etc.), and/or commercially available cell substrates (e.g., CELLstart™ CTS™) and any combination thereof (e.g., a collagen/Matrigel® mixture). In some embodiments, hydrogel from natural polymers, synthetic polymers and hybrid hydrogel can be used to build a scaffold in two dimensions or three dimensions. Examples of natural polymers and synthetic polymers include, but are not limited to, chitosan, agarose, alginate (e.g., AlgiMatrix®), fibrin, silk, polyvinyl alcohol, sodium polyacrylate, acrylate polymers, polyethylene glycol (PEG), synthetic peptides, poly N-isopropylacrylamide, and/or polyacrylamide, and/or any combination thereof. In some embodiments, the surface of a scaffold can be engineered to promote cell adhesion with any one or a combination of ECM molecules, natural or synthetic polymers or synthetic peptides including, but not limited to, poly-l-lysine, RGD-peptide and other integrin recognizing peptide segments. In some embodiments, a cell support structure useful with this presently disclosed subject matter can be mixed with cellular materials (immune cells or other cell types, tissues, blood), or non-cellular materials (drugs, polymer beads, magnetic particles, etc.). In some embodiments, a cell support structure can comprise a two or three dimensional micropatterns or microstructures.

In some embodiments, the cells of the live cell construct can be cultured in liquid medium comprising, for example, an additive, a compound, and/or a solution that can contribute to water balance across the cell layer. In some embodiments, the additive, compound, and/or solution can include, but is not limited to, a hormone, a chemical additive, a food additive, a bacterial metabolite, and/or a hypertonic salt solution. In some embodiments, the additive, compound, and/or solution can be present in/introduced into a luminal reservoir (luminal side of the cell monolayer) and/or into a basal reservoir (basal side of the cell monolayer). Thus, for example, a hormone that stimulates secretion of water and electrolytes to the intestinal lumen can be added to the basal side of the cell monolayer (basal reservoir) to assist in the balance of fluid movement across the cell monolayer. In contrast, food additives and bacterial metabolites can be added to the luminal side of the cell monolayer (luminal reservoir).

In some embodiments, the disclosed live cell constructs, and related methods and systems, can include a culture well or other suitable culture apparatus, culture vessel, system or device as known to those of ordinary skill in the art, including but not limited to a multi-well plate, agar plate, culture dish, vial, tube or the like, for containing the cell support structure, porous membrane and the like, and/or for creating the luminal and/or basal reservoir, as described and shown herein (see, e.g. FIGS. 1 and 3).

In some embodiments, a hormone useful with this presently disclosed subject matter can include, but is not limited to, a vasoactive intestinal peptide (VIP), 5-hydroxytryptamine (serotonin, 5-HT), substance P, bone morphogenetic protein (BMP), gastrin, cholecystokinin, secretin, ghrelin, motilin, gastric inhibitory polypeptide, leptin, glucagon-like peptides, somatostatin, and/or neurotensin.

Non-limiting examples of useful chemical additives include, but are not limited to, butyrate, dibenzazepine, gamma secretase inhibitor (DAPT, LY411575), forskolin, guaifenesin, carbachol, prostaglandins, phorbal ester (phorbol 12-myristate 13-acetate), histamine, and/or N-(1-oxobutyl)-cyclic 3′, 5′-(hydrogen phosphate) 2′-butanoate-adenosine, monosodium salt (i.e., dibutyryl-cAMP, sodium salt) (CAS 16980-89-5).

Exemplary food additives include N-nitrosoanabasine, matairesimol and/or caffeine.

In some embodiments, a bacterial metabolite can include, but is not limited to, a short chain fatty acid.

In some embodiments, a salt in a hypertonic salt solution that is useful with the presently disclosed subject matter can include, but is not limited to, to sodium, chlorine, potassium, magnesium, phosphate, carbonate, and/or lithium. In some embodiments, the concentration of the salt in the hypertonic solution can be about 1 mM to about 1000 mM (e.g., about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 335, 940, 945, 950, 955, 960, 965, 970, 975, 980, 985, 990, 995, 1000 mM, and any value or range therein). Thus, in some embodiments, the concentration of the salt in the solution can be about 5 mM to about 50 mM, about 5 mM to about 100 mM, about 10 mM to about 100 mM, about 10 mM to about 250 mM, about 10 mM to about 500 mM, about 10 mM to about 1000 mM, about 50 mM to about 100 mM, about 50 mM to about 500 mM, about 50 mM to about 1000 mM, about 100 mM to about 250 mM, about 100 mM to about 500 mM, about 100 mM to about 1000 mM, about 200 mM to about 500 mM, about 200 mM to about 1000 mM, about 300 mM to about 500 mM, about 300 mM to about 800 mM, about 300 mM to about 1000 mM, about 400 mM to about 500 mM, about 400 mM to about 800 mM, about 400 mM to about 1000 mM, about 500 mM to about 750 mM, about 500 mM to about 1000 mM, about 600 mM to about 1000 mM, about 700 mM to about 1000 mM, about 800 mM to about 1000 mM, and any value or range therein.

Any substance useful for the growth/maintenance of a cell and/or tissue can be introduced into a basal reservoir or luminal reservoir. In some embodiments, a substance can include, but is not limited to, fibronectin; laminin; epidermal growth factor (EGF); R-spondin; noggin; cytokines (e.g., interleukin (e.g., IL-6, IL-17, IL-22), tumor necrosis factor (TNF)); ephrin receptors (e.g., EphrinB, EphBs); bone morphogenetic proteins (BMIPs, BMP-2, BMP-7); Wnt (wingless-related integration site) (e.g., Wnt3, Wnt3A, and other Wnts); notch signaling factors (notch receptors); Dll1/4; Noggin; Grem1; Grem2; acetate; butyrate; proprionate, desaminotyrosine, catecholamine (e.g., dopamine, norepinephrine) cytokines, and/or short chain fatty acids.

In some embodiments, a method of evaluating the effectiveness of a drug to prevent infection by an organism or to reduce the ability of an organism to infect is provided, comprising: contacting the luminal side of the live cell construct of the presently disclosed subject matter with the organism; contacting the luminal side of the live cell construct with the drug, and determining whether the organism infects one or more cells of the cell monolayer of the live cell construct, wherein the drug is determined to be effective in preventing infection or reducing the ability of an organism to infect if the organism does not infect one or more cells of the cell monolayer of the live cell construct as compared to a control (i.e., contacted with the organism but no drug). In some embodiments, wherein contacting the luminal side of the live cell construct with the organism is prior to, concurrent with, or after contacting the luminal side of the live cell construct with the drug. In some embodiments, a drug can be determined to be effective when about 25% to about 100% of the organisms are killed (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% are killed, and any range or value therein.

In some embodiments, the presently disclosed subject matter provides a method of evaluating an immunological response of a cell monolayer to invasion by an organism, contact by a particle, and or contact by a chemical/compound, comprising: contacting the luminal side of the live cell construct of the presently disclosed subject matter with the organism, particle and or chemical/compound; and assaying cells of the cell monolayer of the live cell construct for the production of a marker associated with an immune response (e.g., a cytokine, a chemokine, a hormone, a neurotransmitter, and/or a antimicrobial peptide), thereby evaluating the immunological response of the cell monolayer of the live cell construct to contact by the organism, particle and or chemical/compound.

In some embodiments, a chemical and/or compound can include, but is not limited to, a dietary metabolite and/or a bacteria metabolite such as vitamins or short chain fatty acids.

An organism that can be studied using the methods and live cell constructs of the present presently disclosed subject matter can be any organism and includes, for example, a bacterium, a virus, a fungus, protozoan, and/or a helminth. Thus, for example, any bacterium, virus, fungus, protozoan, or helminth can be studied for its ability to infect a cell, to evaluate the effectiveness of a drug to prevent infection by the organism/reduce the ability of an organism to infect, and/or to evaluate an immunological response of the cells of the live cell construct in response to contact by the organism.

In some embodiments, the organism can be a bacterium. Non-limiting examples of bacteria include those from the genus Escherichia spp., Yersinia spp., Salmonella spp., Campylobacter spp., Clostridium spp., Helicobacter spp., Bacteroides spp., Peptostreptococcus spp., Vibrio spp., Shigella spp., Salmonella spp., Listeria spp. and Staphylococcus spp. In some embodiments, a bacterium can include, but is not limited to, Acinetobacter baumannii, Actinomyces israelii, Bacillus anthracis, Bacteroides fragilis, Bartonella henselae, Bordetella pertussis, Borrelia burgdorferi, Borrelia garinii, Borrelia afzelil, Borrelia recurrentis, Brucella abortus, Brucella canis, Brucella melitensis, Brucella suis, Burkholderia pseudomallei, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium amycolatum, Corynebacterium diphtheriae, Coxiella burnetii, Ehrlichia canis, Ehrlichia chaffeensis, Enterococcus faecalis, Enterococcus faecium, Escherichia coli, Enterotoxigenic Escherichia coli, Enteropathogenic Escherichia coli, Enteroinvasive Escherichia coli, enterohemorrhagic Escherichia coli, Francisella tularensis, Haemophilus influenzae, Helicobacter pylori, Klebsiella pneumoniae, Legionella pneumophila, Leptospira species, Listeria monocytogenes, Mycobacterium leprae, Mycobacterium tuberculosis, Mycoplasma pneumoniae, Neisseria gonorrhoeae, Neisseria meningitidus, Parachlamydia, Pseudomonas aeruginosa, Nocardia asteroides, Rickettsia rickettsii, Salmonella bongori, Salmonella enterica, Staphylococcus aureus, Staphylococcus epidermidis, Staphylococcus saprophyticus, Streptococcus agalactiae, Streptococcus pneumoniae Streptococcus pyogenes, Streptococcus viridans, Treponema pallidum, Vibrio cholerae, Vibrio vulnificus, Vibrio parahaemolyticus, and/or Yersinia pestis.

In some embodiments, the organism can be a protozoan. Non-limiting examples of protozoa include those from the phylum of Amoebozoa, Excavata, and/or Chromalveolata. In some embodiments, a protozoan can include, but is not limited to, those from the genus Amoeba spp., Entamoeba spp., Plasmodium spp., Giardia spp., and/or Trypanosoma spp. In some embodiments, a protozoan can include, but is not limited to, Entamoeba histolytica, Cryptosporidium parvum, Cryptosporidium hominis, Cyclospora cayetanensis, and/or Giardia lamblia

In some embodiments, the organism can be a virus. Non-limiting examples of viruses include Simplexvirus, Varicellovirus, Cytomegalovirus, Roseolovirus, Lymphocryptovirus, Rhadinovirus, Adenovirus, Astrovirus, Calicivirus, Mastadenovirus, Alphapapillomavirus, Betapapillomavirus, Gammapapillomavirus, Mupapillomavirus, Nupapillomavirus, Polyomavirus, Molluscipoxvirus, Orthopoxvirus, Parapoxvirus, Alphatorquevirus, Betatorquevirus, Gammatorquevirus, Gemycircularviruses, Erythrovirus, Dependovirus, Bocavirus, Coltivirus, Rotavirus, Seadornavirus, Hepevirus, Alphacoronavirus, Betacoronavirus, Torovirus, Mamastrovirus, Norovirus, Sapovirus, Flavivirus, Hepacivirus, Pegivirus, Cardiovirus, Cosavirus, Enterovirus, Hepatovirus (e.g., hepatitis A), Kobuvirus, Parechovirus, Rosavirus, Salivirus, Alphavirus, Rubivirus, Deltavirus, Lyssavirus, Vesiculovirus, Filoviridae, Ebolavirus, Marburgvirus, Paramyxoviridae, Henipavirus, Morbilivirus, Respirovirus, Rubulavirus, Metapneumovirus, Pneumovirus, Arenavirus, Peribunyaviridae, Orthobunyavirus, Hantavirus, Nairovirus, Phenuiviridae, Phlebovirus, Influenzavirus A Influenzavirus B, Influenzavirus C, Thogotovirus, Gammaretrovirus Deltaretrovirus, Lentivirus, Spumavirus, and/or Orthohepadnavirus,

In some embodiments, the organism can be a helminth including, but not limited to, intestinal flukes, round worms, pin worms, and/or tape worms. In some embodiments, the helminth can include, but is not limited to, a helminth from the genus of Ascaris spp., Ancylostoma spp., Trichuris spp, Strongyloides spp., Necator spp., Schistosoma spp., and/or Trichinella spp. Further non-limiting examples of helminths include Ascaris lumbricoides (roundworm), Ancylostoma duodenale (hookworm), Necator americanus (hookworm), Strongyloides stercoralis, Trichinella spiralis and/or Trichuris trichiura (whipworm).

In some embodiments, the organism can be a fungus. Non-limiting examples of fungi include those from the genus Candida spp., Aspergillus spp., Mucor spp., Fusarium spp., Blastomyces spp., Coccidioides spp., Cryptococcus spp., Histoplasma spp., Rhizopus spp., Lichtheimia spp., Pneumocystis spp., Sporothrix spp. and/or Cunninghamella spp. Further non-limiting examples of fungi include Candida albicans, Candida tropicalis, Aspergillus flavus, Aspergillus fumigatus, Aspergillus niger, Cryptococcus neoformans, Cryptococcus gattii, Pneumocystis jirovecii, and/or Torulopsis, glabrata.

The presently disclosed subject matter will now be described with reference to the following examples. It should be appreciated that these examples are not intended to limit the scope of the claims to the presently disclosed subject matter, but are rather intended to be exemplary of certain embodiments. Any variations in the exemplified methods that occur to the skilled artisan are intended to fall within the scope of the presently disclosed subject matter.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

Traditional Monolayer Culture Method is Unable to Generate Sufficient EEC and EC Cells for Reliable Hormone Assay

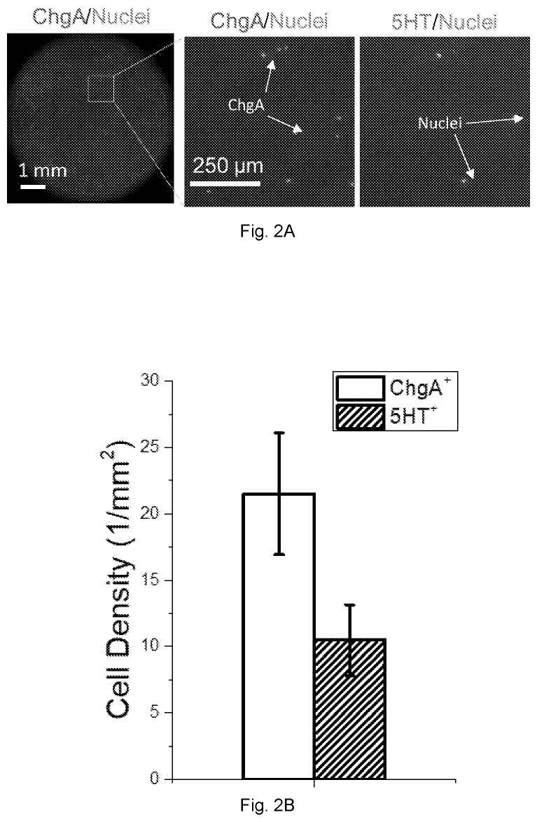

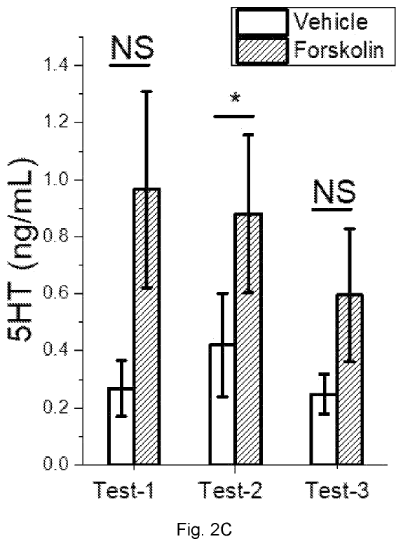

Culture conditions for intestinal epithelial monolayer platform have been optimized in a previous publication, and the medium composition for yielding the maximal expression levels of EEC cell lineage marker gene ChgA was investigated.¹¹ Nicotinamide and p38 MAP kinase inhibitor (SB202190), two compounds commonly used in intestinal epithelial cell culture, are removed from medium due to their suppression of differentiation towards secretory cells. Inclusion of A 8301, a TGF-β receptor signaling inhibitor, is required for proper differentiation and cell morphology of monolayer cultures. Herein, the same medium composition was adopted and the in vitro monolayers were generated from stem cells derived from human transverse colon using the traditional submerged culture. The EEC (ChgA+) and EC (5HT+) cells presented at a density of 22±5 and 10±3 mm⁻², respectively (FIG. 2A, 2B). Accordingly, the total number of EC cells in a 96-well insert (surface area=11 mm²) is 110±33. To test if the number of EC cells is enough for functional assays, the monolayers were stimulated with vehicle (dimethyl sulfoxide [DMSO]) or forskolin (10 μM) at the apical side for 4 h, and the released 5HT from the basal side was collected and quantified (FIG. 2C). Forskolin is a potent stimulus of 5HT release though activation of the cAMP-dependent pathway.¹⁸ In three independent experiments, stimulated 5HT secretion by forskolin was 0.97±0.34, 0.88±0.28, and 0.60±0.23 ng/mL, respectively, corresponding to 3.6×, 2.1× and 2.4× induction over the vehicle controls (0.27±0.10, 0.42±0.18 ng/mL, and 0.25±0.07 respectively). Significant inter-experiment variability was observed due to high standard deviation of the 5HT measurements: the results in the first experiment were considered to be statistically non-significant (p>0.05, t-test), statistically significant (p<0.05) in the second one, but statistically non-significant (p>0.05) again in the third experiment. The high standard deviation came from a number of sources: error in 5HT measurement through enzyme-linked immunosorbent assay (ELISA), well-to-well variation, 5HT residue (mainly from the fetal bovine serum in the medium) left in the well, and 5HT that spontaneously leached out from the apoptotic ECs during assay. To improve the reliability of 5HT secretion assay and its signal/background ratio, the number of EEC and EC cells in monolayers need to be enriched.

### Example 2

### Enriching EEC and EC Cells by a Novel VIP-Assisted ALI Culture Method and System

IESCs and their differentiated cells are generally maintained in the submerged culture. Recently, air-liquid interface (ALI) culture has been recently applied to intestinal monolayer cultures to improve the secretory cell lineage differentiation with focus on goblet cells and mucus formation.¹⁹⁻²¹ Being different from traditional submerged culture, the apical surface of monolayers in ALI culture was exposed to air and completely dry. Notably, this situation does not reflect the in vivo intestinal luminal environment due to the absence of a high water content at the apical surface. Indeed, water and electrolyte homeostasis of the colonic mucosa are balanced with water moving into and out of the lumen. To improve the ALI culture, the present disclosure used an intestinal hormone, vasoactive intestinal peptide (VIP), to assist in the balance of fluid movement across the epithelium so that a thin layer of fluid (about 0.1 mm to about 1.0 mm, or about 0.4 mm in thickness) was maintained at the apical reservoir to prevent the dry up. This VIP-assisted ALI culture method significantly boosted the formation of goblet cells and assisted the mucus secretion/accumulation. Since both goblet and EEC cells belong to the secretory cell lineage, it is likely that these simple methods can increase the presence of EEC and EC cells in the in vitro monolayer system.

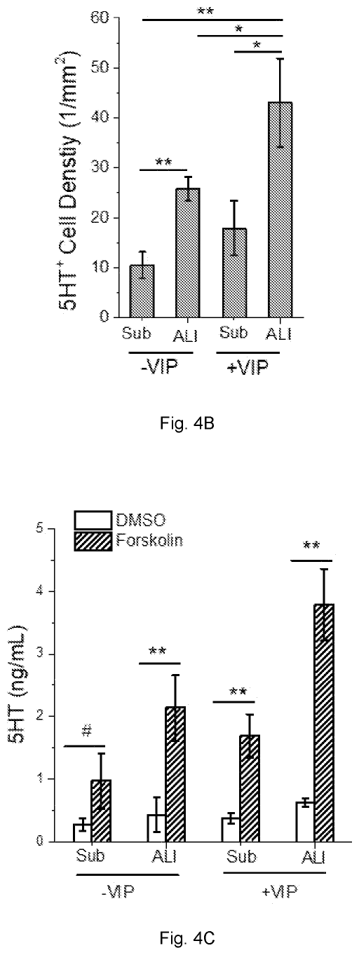

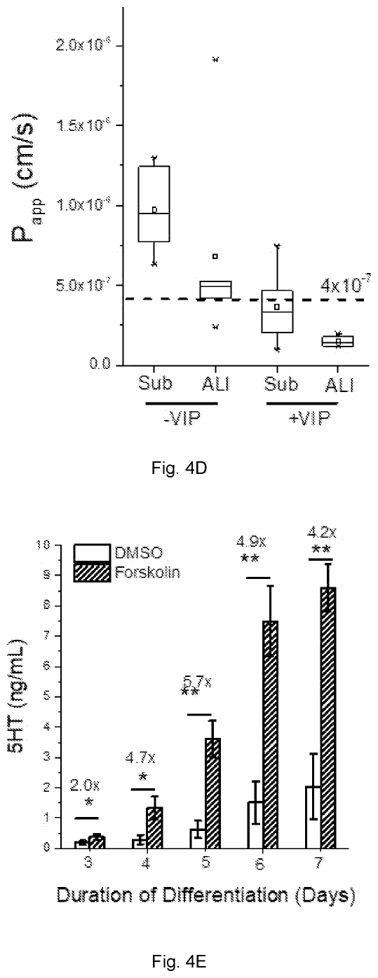

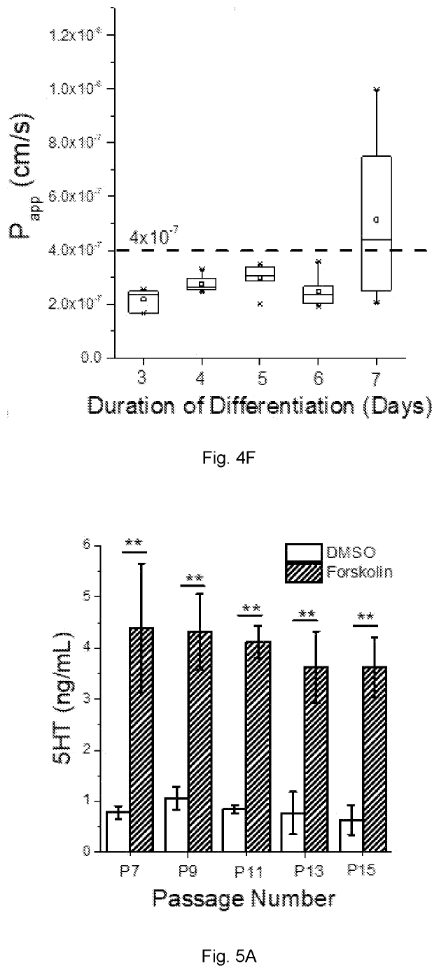

To verify if ALI culture and VIP improve the EEC lineage differentiation, the colonic monolayers derived from human transverse colon were differentiated under four strategies outlined in FIG. 3A, and the presence of EEC and EC cells in monolayers were revealed by immunofluorescence staining (FIG. 3B). Compared with submerged culture (EEC: 22±5, EC: 10±3 mm⁻²), ALI alone significantly increased the number of EEC (259%) and EC (260%). Addition of VIP to submerged culture also increased the number of EEC (177%) and EC (180%), but to a less extent compared with ALI. A synergistic effect was observed by a combination of ALI and VIP, which significantly increased the number of EEC (377%) and EC (430%) compared with the traditional submerged culture without VIP (FIGS. 4A and 4B). The monolayers possessed an enriched density of EEC (83±11) and EC (43±9 mm⁻²). The fraction of EC among EEC remained in the range of 45-51% in these differentiation conditions, which is consistent with their fraction in the body that EC make up over 70% of the EEC population in the proximal colon and falls to around 40% in the rectum.² To test if the enriched EC cells increases 5HT secretion, the monolayers were stimulated with vehicle (DMSO) or forskolin (10 μM). The 5HT secretion was found to correlate with the number of EC cells (FIG. 4C). The monolayer generated from VIP-assisted ALI culture had a boosted 5HT secretion under forskolin stimulation (3.79±0.57 ng/mL), which is 390% higher than that generated from the submerged culture (0.97±0.44 ng/mL). The induction is 6.1× over the vehicle control (0.62±0.07 ng/mL). Therefore, the enriched EC cells improves the 5HT secretion assay by increasing the signal/background ratio.

### Example 3

### VIP-Assisted ALI Culture Generated Monolayers with High Barrier Integrity

Besides the elevated 5HT secretion, an additional advantage of VIP-assisted ALI culture is the improved barrier integrity (FIG. 4D). Among four differentiation strategies, only VIP-assisted ALI culture generated monolayers possessing acceptable barrier integrity with low permeability coefficients (Pₐₚₚ<4×10⁻⁷ cm/s, n=6) and high transepithelial electrical resistance (TEER, 842±166 ohms cm², n=6). Submerged culture (in the absence or presence of VIP) failed to generate contiguous monolayers probably due to the oxygen gradient in the growth medium. The cell respiration combined with the low diffusion rate of oxygen in the growth medium can lead to a reduced oxygen availability to the cells.²² ALI culture can increase the oxygen availability, but the cells were stressed due to the complete dry up at the apical surface. As a result, ALI culture failed to generate contiguous monolayers. Conversely, VIP-assisted ALI culture maintained a thin layer of fluid (about 0.4 mm thick) at the apical side, thus increasing the oxygen availability and at the same time as eliminating the stress from dry up. The high integrity of monolayers is needed to effectively segregates the apical and basal reservoirs to prevent the leakage of the stimulants and 5HT.

As the generation and storage of 5HT inside granules of EC cells depends on the cell maturation, the level of 5HT secretion is affected by the differentiation length of the monolayers. To find out the optimal duration for differentiation, the monolayers were subjected to VIP-assisted ALI culture for 3-7 days. As expected, the forskolin stimulated 5HT secretion increased with differentiation length (due to cell maturation), so did the non-stimulated basal secretion (FIG. 4E). The barrier integrity was remained (Pₐₚₚ<4×10⁻⁷ cm/s) for all samples, except for those under 7 days in differentiation in which the cells may start to undergo apoptosis (FIG. 4F). 5 days in differentiation was considered to be optimal since it generated monolayers with high 5HT induction (5.7×) and monolayer barrier integrity. This duration is coincident with life span of differentiated epithelium in human colon. Unless otherwise specified, 5 days were used for differentiation in this invention.

### Example 4

### Passage and Donor Variations

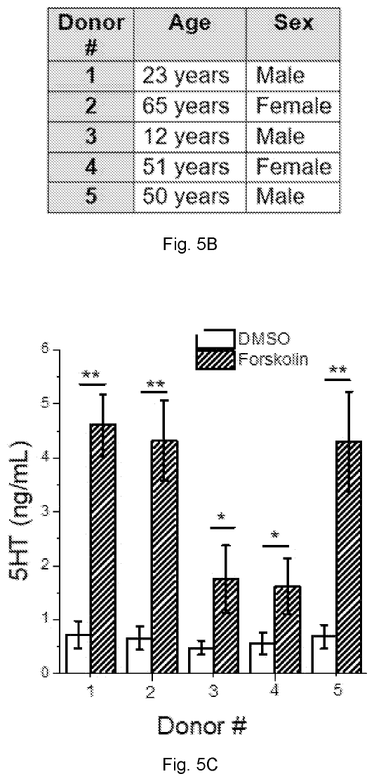

Stem cell passage number is an important consideration when designing the cell-based assay as they are vulnerable to replicative senescence when multiplying them in vitro.²³ To study if the passage number affect the 5HT secretion, the stem cells from human transverse colon were plated on the inserts at different passage numbers (7, 9, 11, 13 and 15). No significant variation of 5HT secretion level (both vehicle and forskolin stimulated) was observed (FIG. 5A). The induction of 5HT secretion (forskolin/DMSO) was in the range of 4.1-5.7×. The stem cells after passage number 16 were discarded due to the possible chromosomal abnormalities.

Primary stem cells derived from different donors are known to behave differently in culture conditions depending on the age, gender, and health of individuals from whom the tissue was derived.²⁴ To study the donor variation, the stem cells of transverse colon from 5 cadaveric donors at different ages and genders (FIG. 5B) were used to investigate the variation of cell behavior in terms of 5HT secretion. Significant variation of 5HT secretion level (both vehicle and forskolin stimulated) was observed (FIG. 5C). Low 5HT induction (forskolin/DMSO, <3.6) was observed for donor 3 and 4, while high 5HT induction (>5.0) for donor 1, 2, and 5. Significant donor variation suggests that 5HT secretion behavior is complicated and may be associated with many other factors besides gender and age, such as body mass index, blood sugar level, dietary habit and health status. Therefore, a full investigation of donor variation needs to be established for primary cell based assay. As the cells from donor 2 has the mostly representative behavior, they were used for the above studies and the subsequent screening experiment.

### Example 5

### Small Scale Screening of Pungent Food Ingredients

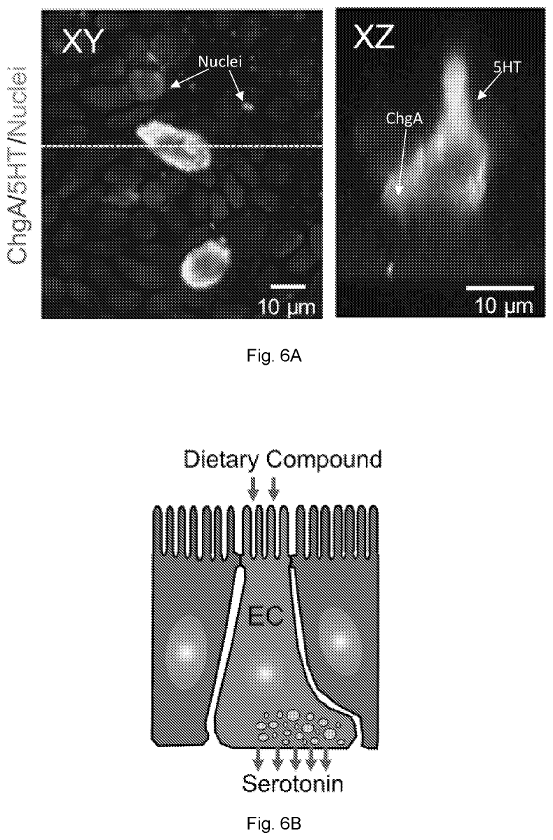

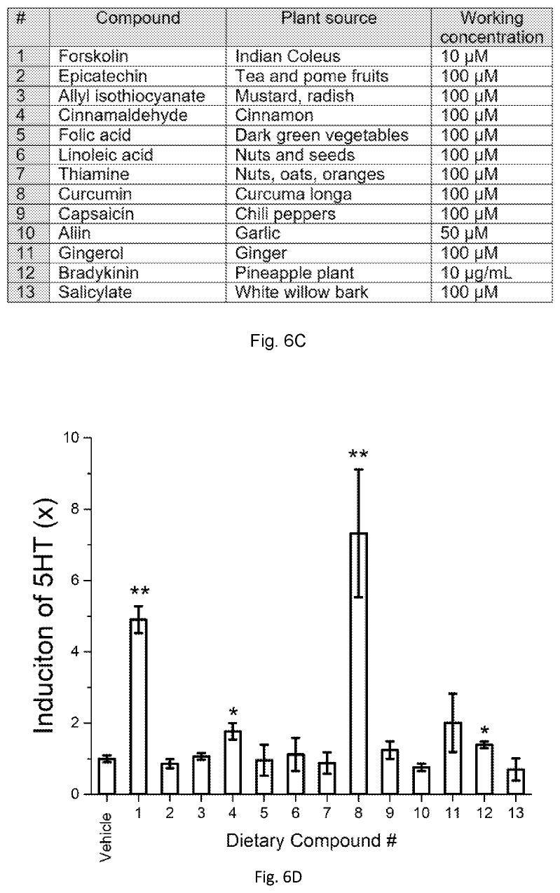

In vivo, EC cells are characterized by a pyramidal shape with a large basolateral surface. This pyramidal shape was recreated in the in vitro monolayer platform (FIG. 6A). Like other EEC subtypes, EC cells sense luminal contents, particularly dietary compounds and metabolites, through cell surface sensory receptors, and release 5HT through basolateral border into the lamina propria (FIG. 6B).⁷ The EC-enriched, contiguous monolayers allow independent stimulate and analyze the EC response at the apical and/or basal sides. This is an advantage over the widely used intestinal organoid systems which has an inaccessible lumen. To demonstrate the utility of the monolayer system, 13 types of pungent plant food ingredients (FIG. 6C) were tested for their ability to modulate 5HT production in response to luminal stimuli (FIG. 6D). Among these compounds, curcumin and forskolin were found to be the most potent ones with 5HT induction of 7.3× and 4.9×, respectively. Cinnamaldehyde and bradykinin slightly increase the 5HT secretion, with induction of 1.8× ad 1.4×, respectively. Curcumin stimulated the 5HT secretion in a dose-dependent manner with a median effective dose (E50) of 51 μM. Other compounds didn't significantly stimulate the secretion of 5HT.

### Example 6

### Presence of Other EEC Subtype

Enteroendocrine (EEC) cells within the intestinal epithelium consist of at least 15 different subtypes. Enterochromaffin (EC) cells are the most abundant subtype, comprising about 40% of EEC cells in the gastrointestinal tract. Besides EC cells, at least one other important EE subtype is L-cells, which are major regulators of the gut-brain interactions and are pivotal in regulating appetite and glucose homeostasis⁴. L-cells co-secrete peptide hormones GLP-1 and PYY in response to the ingestion of food⁵⁻⁸. GLP-1 stimulates the secretion of insulin from pancreatic β-cells^{9,10}, decreases gastric emptying and increases satiation¹¹. PYY slows GI motility and reduces food intake¹². Due to their important function as regulators of postprandial response to nutrients, L-cells are of specific interest in the field of obesity and diabetes³⁻¹⁷. L-cells were also identified in the VIP-ALI cultured monolayers at a density of 12±2 mm² and constituted about 14% of the EE population (FIG. 7). However, the density of L-cells is still too low to perform a reliable functional assay of GLP-1/PYY secretion. Thus, further experiments were conducted to develop optimal protocols to enrich L-cells (see Example 7).

### Example 7

### Optimization of Enteroendocrine L-Cell Formation in Stem-Cell Derived Monolayer System to Enable Hormone Secretion Assay

Optimization of Differentiation Medium and Culture Format to Enrich L-Cells.

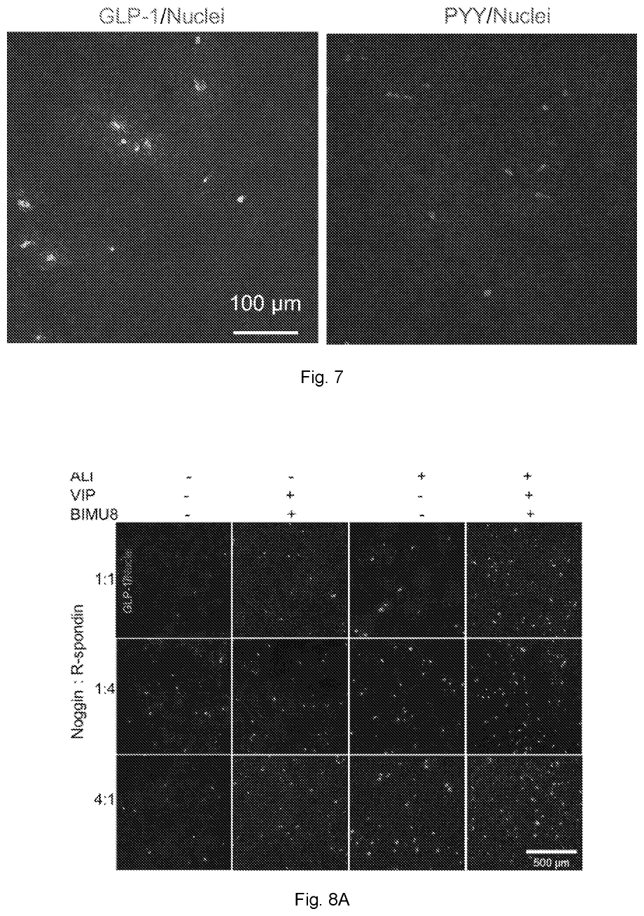

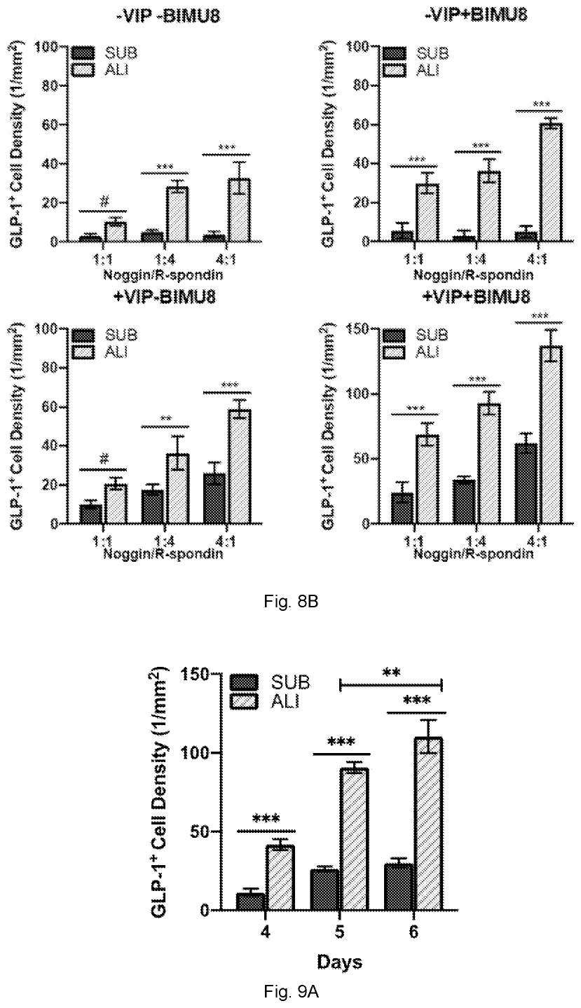

Experiments were conducted to systemically optimize four conditions to enrich L-cells: (1) ±ALI, (2) ±VIP, (3) R-spondin/Noggin ratio at 1:4, 1:1 and 4:1 in differentiation medium, (4) ±BIMU8 (a potent 5-HT4 agonist). The colonic stem cells were plated on a porous membrane coated with Matrigel and cultured for 3 days in growth factor-enriched medium until the cell monolayer reach confluence. Then the cells were differentiated for 4 days and the density of GLP-1⁺ L cells was used as the readout (FIG. 8A). As expected, based on the data presented herein, in all conditions, ALI culture significantly increased the formation of L-cells relative to that of the submerged culture conditions. Addition of VIP and BIMU8 during differentiation also increased the formation of L-cells. See FIG. 8B. The R-spondin/Noggin ratio in differentiation medium also affected the density of L-cells, and the best result was obtained by using a ratio of 4/1. By using the conditions disclosed herein (ALI, VIP, R-spondin/Noggin=4/1, BIMU8), 137±12 cells/mm² GLP-1⁺ L-cells were obtained, which is about 46 times higher than 3±2 cells/mm² in the previously used conditions (submerged, —VIP, R-spondin/Noggin=1/1, -BIMU8), and 11 times higher than 12±2 mm² in VIP-ALI culture (+ALI, +VIP, R-spondin/Noggin=1/1, -BIMU8).

Based on this research, the addition of other compounds, chemicals, hormones and the like to medium in the disclosed systems and methods can be used to further enhance EEC and EC cell growth, and particularly L-cell development. Further non-limiting examples of compounds and hormones are listed in Table 2 below.

TABLE 2

| | Range of | | |
|---|---|---|---|
| Reagent | Conc. (μM) | REF. | Description |
| Forskolin | 0.1-100 | 40 | Increase levels of cyclic AMP |
| Isobutylmethylxanthine | 0.1-100 | 40 | Raises intracellular cAMP |
| (IBMX) | | | |
| Oligofructose | 0.01-10 | 41 | Stimulate cell proliferation in the colon linked with |
| Inulin-type fructans | 0.1-100 | 41 | increased enteroglucagon plasma levels. |
| Serotonin | 0.001-1 | 42 | Paracrine stimulation from neighboring |
| | | | enterochromaffin cells promoting L-cells formation |
| Arginine vasopressin | 0.1-100 | 43 | Promote retention of water by increasing permeability |
| | | | of the epithelium |
| Angiotensin II | 0.1-100 | 43 | Strong vasopressor activity, bind and interact with |
| | | | specific G protein coupled receptors. |
| BIMU8 | — | 42 | Induced activation of serotonin 5-hydroxytryptamine |
| | | | receptor 4 (5-HT4) signaling |
| Liraglutide | 0.1-100 | 44 | Improved glucose tolerance and insulin response. |
| | | | Antihyperglycemic activity |
| FFAR1 agonist | 0.1-100 | 43 | Promotes the secretion of peptide YY |
| GPR119 agonist | 0.1-100 | 42, | Activates the Gs signaling pathway resulting in |
| | | 43, | increased cAMP |
| | | 45 | |
| GPBAR1 agonist | 0.1-100 | 42, | Mimicked by downstream serotonin signaling |
| | | 43, | conceivably through paracrine stimulation from |
| | | 45 | neighboring enterochromaffin cells |
| IL-1β | 0.1-100 | 46 | Regulated intestinal homeostasis and immune responses |
| IL-6 | 0.01-10 | 46 | regulation activating EEC inhibiting histone acetylation |
| TNF-α | 0.1-100 | 46 | enzymes or GPCRs pathway. |
| Acetate | 0.001-1 | 47 | Play a role in energy and metabolism regulation. |
| Propionate | 0.1-100 | 47 | Butyrate is the ligand for metabolite-sensing G-protein |
| Butyrate | 0.1-100 | 47, | coupled receptors (GPCRs), such as GPR43, GPR41 |
| | | 48 | and GPR109A |
| LPS | — | 46 | LPS could act through a TLR4 receptor located on the |
| | | | basolateral membranes of EECs with resultant GLP-1 |
| | | | secretion |
| Akkermansia muciniphila, | 0.1-100 | 41, | Regulated intestinal bioactive lipids involved in |
| Bifidobacterium spp., | | 48 | enteroendocrine peptides secretion |
| Lactobacillus spp. | | | |

Additional compounds suitable for addition to a medium in the disclosed systems and methods to increase L-cell formation include, but are not limited to: short chain fatty acids^{8,11}; oligofructose¹⁰; secondary bile acids, vegetable-derived compounds; L-amino acids Phe, Trp, Gln and Ala; dipeptide glycine-sarcosine¹¹.

The addition of any such compounds, hormones or the like to the disclosed culture methods and systems can in some embodiments enhance EEC and EC cell growth, and particularly L-cell development.

### References for Above Information:

Improving the Differentiation Duration and Quantification of Both GLP-1 and PYY Markers.

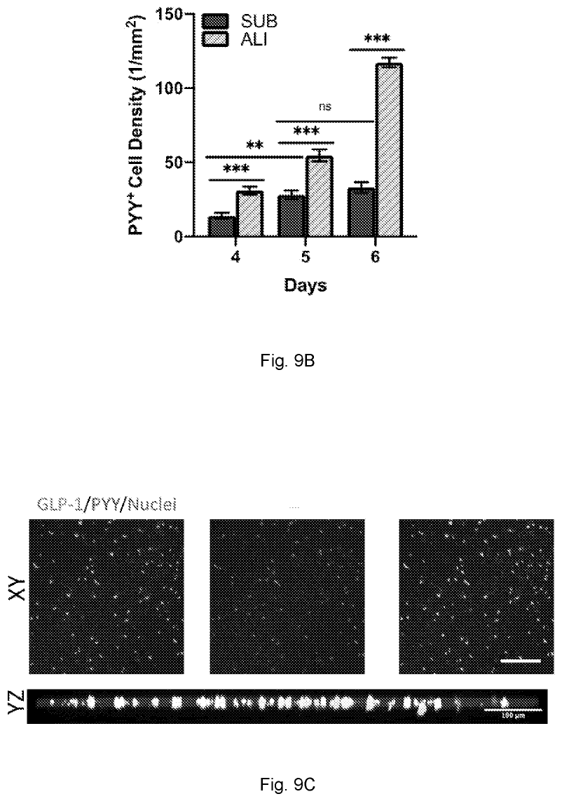

The differentiation duration (4-6 days) was also improved to identify the best time to maximize L-cell number and perform the hormone secretion assay (FIGS. 9A and 9B). Six days in differentiation generated the most abundant L-cells in terms of GLP-1 and PYY markers (FIG. 9C). Not all L-cells are co-stained with both GLP-1 and PYY. 86% GLP-1⁺ cells are co-located with PYY, and 81% PYY⁺ cells are co-located with GLP-1. The cross-sectional image (FIG. 9C, bottom) shows the location of L-cells in a contiguous monolayer. The accessible apical of the L-cells enables stimulation from the apical monolayer side (i.e. replicating physiologic stimulation). Hormones (GLP-1 and PYY) are then secreted from the basal side of the monolayer. The high resistance of the monolayer enables the monolayer to act as an impermeable barrier so that secreted hormone remains in the basal reservoir and is not diluted across both the apical and basal reservoirs. This further improves detection limits. The accessible basal side of the monolayer permits sampling of the basal reservoir with quantification of the secreted hormone.

Hormone GLP-1 Secretion Assay from the L-Cell Platform.

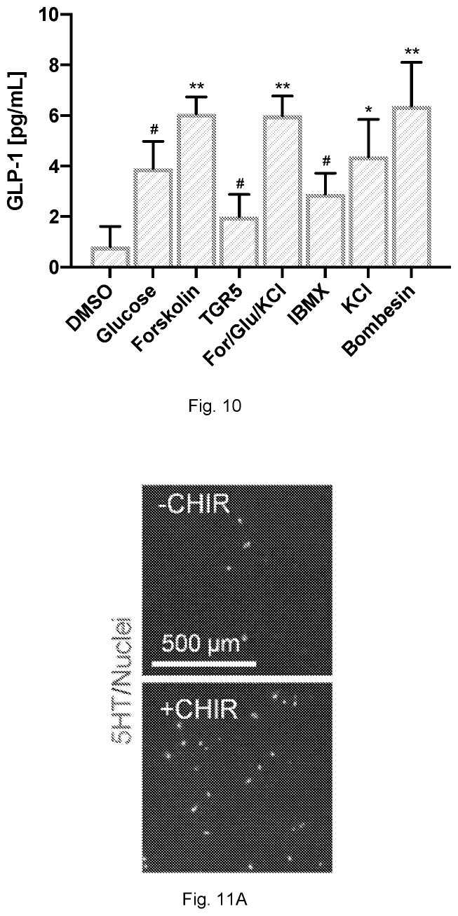

To validate the platform for a hormone secretion assay, the optimized monolayers were used to study the secretion of hormone (GLP-1) under a number of apical stimuli. The apical surface of the differentiated colonic monolayers was exposed for 4 hours to 7 different stimuli that have the potential to stimulate GLP-1 secretion. The media at the basal side were collected for an enzyme-linked immunosorbent assay (ELISA) of GLP-1. Forskolin and bombesin were the most potent in inducing GLP-1 secretion, about six times higher than the control (DMSO). These data (FIG. 10) demonstrate the power of using this system for screening of drugs, bacteria and other microbes, food stuff, microbe and food metabolites as well as other chemicals for their impact on L cell hormone secretion.

### Example 8

### Increase EEC and EC Cells by Other Approach: Preventing the Early Lineage-Fate Decision of Stem Cells

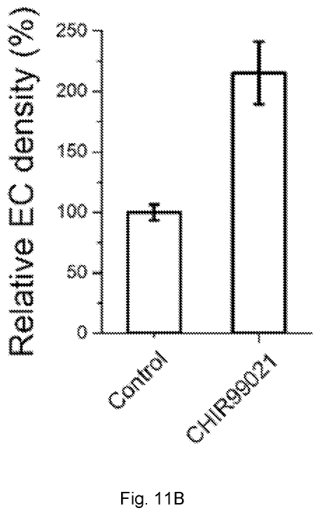

It is likely that stem cells make lineage-fate decisions at the proliferation stage toward enterocytes and goblets cells, which are the major cell types in the intestinal epithelium. We can add signaling molecules, such as CHIR99021, to prevent the early lineage-fate decision and force the stem cells to stay at their ground state. CHIR99021 activates Wnt signaling by inhibition of GSK3. When the stem cells were cultured in EM under 3 μM CHIR99021 prior to conventional submerged culture in DM, the presence of EC cells was increased by almost 200% (FIGS. 11A and 11B). This result suggests that activation of Wnt signaling prevents early lineage-fate decision. Besides CHIR99021, other Wnt activators (WAY316606, ABC99, IQ1, arylpyrimidine), and recombinant proteins of Wnt-3A and R-spondin (Wnt signaling enhancer) may be able to increase the formation of EC cells. We have not studied the synergistic effect of Wnt activator (in EM) and VIP-assisted ALI (in DM), but we expect the combination of both strategies will further increase the formation of EEC and EC cells.

### Discussion of Examples 1-8

The present disclosure, including the above examples, illustrate a new strategy and system, described herein as VIP-assisted ALI culture, to significantly boost the number of EEC and EC cells over the traditional submerged culture, while at the same time maintaining a high barrier integrity of monolayers. The new strategy, systems and devices overcome the limitations of the existing EEC enrichment methods by maintaining high cell viability and barrier integrity and without requiring complicated procedures of cocultures or genetic engineering/induction. The created EEC-enriched, contiguous monolayer platform acts as a robust analytical tool to enable functional studies of hormone secretion from EEC cells with high signal background ratio and repeatability.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Latorre R, Sternini C, De Giorgio R, Greenwood-Van Meerveld B. Enteroendocrine cells: a review of their role in brain-gut communication. Neurogastroenterology and Motility 2016; 28:620-630.
2. Gunawardene A R, Corfe B M, Staton C A. Classification and functions of enteroendocrine cells of the lower gastrointestinal tract. International Journal of Experimental Pathology 2011; 92:219-231.
3. Chang-Graham A L, Danhof H A, Engevik M A, Tomaro-Duchesneau C, Karandikar U C, Estes M K, Versalovic J, Britton R A, Hyser J M. Human Intestinal Enteroids With Inducible Neurogenin-3 Expression as a Novel Model of Gut Hormone Secretion. Cellular and Molecular Gastroenterology and Hepatology 2019; 8:209-229.
4. Braun T, Voland P, Kunz L, Prinz C, Gratzl M. Enterochromaffin cells of the human gut: Sensors for spices and odorants. Gastroenterology 2007; 132:1890-1901.
5. Bellono N W, Bayrer J R, Leitch D B, Castro J, Zhang C C, O'Donnell T A, Brierley S M, Ingraham H A, Julius D. Enterochromaffin Cells Are Gut Chemosensors that Couple to Sensory Neural Pathways. Cell 2017; 170:185-198.
6. Kidd M, Gustafsson B I, Drozdov I, Modlin I M. IL1β- and LPS-induced serotonin secretion is increased in E C cells derived from Crohn's disease. Neurogastroenterology & Motility 2009; 21:439-450.
7. Lund M L, Egerod K L, Engelstoft M S, Dmytriyeva O, Theodorsson E, Patel B A, Schwartz T W. Enterochromaffin 5-H T cells—A major target for GLP-1 and gut microbial metabolites. Molecular Metabolism 2018; 11:70-83.
8. Sato T, Vries R G, Snippert H J, van de Wetering M, Barker N, Stange D E, van Es J H, Abo A, Kujala P, Peters P J, Clevers H. Single Lgr5 stem cells build crypt-villus structures in vitro without a mesenchymal niche. Nature 2009; 459:262-U147.
9. Sato T, Stange D E, Ferrante M, Vries R G J, van Es J H, van den Brink S, van Houdt W J, Pronk A, van Gorp J, Siersema P D, Clevers H. Long-term Expansion of Epithelial Organoids From Human Colon, Adenoma, Adenocarcinoma, and Barrett's Epithelium. Gastroenterology 2011; 141:1762-1772.
10. Wang Y, DiSalvo M, Gunasekara D B, Dutton J, Proctor A, Lebhar M S, Williamson I A, Speer J, Howard R L, Smiddy N M, Bultman S J, Sims C E, Magness S T, Allbritton N L. Self-renewing monolayer of primary colonic or rectal epithelial cells. Cellular and Molecular Gastroenterology and Hepatology 2017; 4:165-182.
11. Kozuka K, He Y, Koo-Mccoy S, Kumaraswamy P, Nie B, Shaw K, Chan P, Leadbetter M, He L, Lewis J G, Zhong Z, Charmot D, Balaa M, King A J, Caldwell J S, Siegel M. Development and Characterization of a Human and Mouse Intestinal Epithelial Cell Monolayer Platform. Stem Cell Reports 2017; 9:1976-1990.
12. VanDussen K L, Marinshaw J M, Shaikh N, Miyoshi H, Moon C, Tarr P I, Ciorba M A, Stappenbeck T S. Development of an enhanced human gastrointestinal epithelial culture system to facilitate patient-based assays. Gut 2015; 64:911-920.
13. Moon C, Vandussen K L, Miyoshi H, Stappenbeck T S. Development of a primary mouse intestinal epithelial cell monolayer culture system to evaluate factors that modulate IgA transcytosis. Nature Mucosal Immunol 2014; 7:818-28.
14. Noel G, Baetz N W, Staab J F, Donowitz M, Kovbasnjuk O, Pasetti M F, Zachos N C. A primary human macrophage-enteroid co-culture model to investigate mucosal gut physiology and host-pathogen interactions. Scientific Reports 2017; 7:45270.
15. In J, Foulke-Abel J, Zachos N C, Hansen A-M, Kaper J B, Bernstein H D, Halushka M, Blutt S, Estes M K, Donowitz M, Kovbasnjuk O. Enterohemorrhagic Escherichia coli Reduces Mucus and Intermicrovillar Bridges in Human Stem Cell-Derived Colonoids. Cellular and Molecular Gastroenterology and Hepatology 2016; 2:48-62.e3.
16. Puzan M, Hosic S, Ghio C, Koppes A. Enteric Nervous System Regulation of Intestinal Stem Cell Differentiation and Epithelial Monolayer Function. Scientific Reports 2018; 8:6313.
17. Basak O, Beumer J, Wiebrands K, Seno H, van Oudenaarden A, Clevers H. Induced Quiescence of Lgr5+Stem Cells in Intestinal Organoids Enables Differentiation of Hormone-Producing Enteroendocrine Cells. Cell Stem Cell 2017; 20:177-190.
18. Tinajero J C, Fabbri A, Ciocca D R, Dufau M L. Serotonin secretion from rat Leydig cells. Endocrinology 1993; 133:3026-3029.
19. Wilke G, Funkhouser-Jones L J, Wang Y, Ravindran S, Wang Q L, Beatty W L, Baldridge M T, VanDussen K L, Shen B, Kuhlenschmidt M S, Kuhlenschmidt T B, Witola W H, Stappenbeck T S, Sibley L D. A Stem-Cell-Derived Platform Enables Complete Cryptosporidium Development In Vitro and Genetic Tractability. Cell Host & Microbe 2019; 26:123-134.
20. Wang Y, Kim R, Sims C E, Allbritton N L. Building a Thick Mucus Hydrogel Layer to Improve the Physiological Relevance of In Vitro Primary Colonic Epithelial Models. Cellular and Molecular Gastroenterology and Hepatology 2019:doi: 10.1016/j.jcmgh.2019.07.00.
21. Wang Y, Chiang I L, Ohara T E, Fujii S, Cheng J, Muegge B D, Ver Heul A, Han N D, Lu Q, Xiong S, Chen F, Lai C-W, Janova H, Wu R, Whitehurst C E, VanDussen K L, Liu T-C, Gordon J I, Sibley L D, Stappenbeck T S. Long-Term Culture Captures Injury-Repair Cycles of Colonic Stem Cells. Cell 2019; 179:1144-1159.e15.
22. Place T L, Domann F E, Case A J. Limitations of oxygen delivery to cells in culture: An underappreciated problem in basic and translational research. Free Radical Biology and Medicine 2017; 113:311-322.
23. Tan Q, Lui P P Y, Rui Y F. Effect of in vitro passaging on the stem cell-related properties of tendon-derived stem cells-implications in tissue engineering. Stem cells and development 2012; 21:790-800.
24. Ketterl N, Brachtl G, Schuh C, Bieback K, Schallmoser K, Reinisch A, Strunk D. A robust potency assay highlights significant donor variation of human mesenchymal stem/progenitor cell immune modulatory capacity and extended radio-resistance. Stem cell research & therapy 2015; 6:236-236
25. Israelyan, N.; Del Colle, A.; Li, Z.; Park, Y.; Xing, A.; Jacobsen, J. P. R.; Luna, R. A.; Jensen, D. D.; Madra, M.; Saurman, V.; Rahim, R.; Latorre, R.; Law, K.; Carson, W.; Bunnett, N. W.; Caron, M. G.; Margolis, K. G. Effects of Serotonin and Slow-Release 5-Hydroxytryptophan on Gastrointestinal Motility in a Mouse Model of Depression. Gastroenterology 2019. https://doi.org/10.1053/j.gastro.2019.04.022.
26. Wang, Y.; Sims, C. E.; Allbritton, N. L. Enterochromaffin Cell-Enriched Monolayer Platform for Assaying Serotonin Release from Human Primary Intestinal Cells. Anal. Chem. 2020. https://doi.org/10.1021/acs.analchem.0c02016.
27. Spreckley, E.; Murphy, K. G. The L-Cell in Nutritional Sensing and the Regulation of Appetite. Frontiers in Nutrition. 2015. https://doi.org/i0.3389/fnut.2015.00023.
28. Lewis, J. E.; Miedzybrodzka, E. L.; Foreman, R. E.; Woodward, O. R. M.; Kay, R. G.; Goldspink, D. A.; Gribble, F. M.; Reimann, F. Selective Stimulation of Colonic L Cells Improves Metabolic Outcomes in Mice. Diabetologia 2020. https://doi.org/10.1007/s00125-020-05149-w.
29. De Silva, A.; Salem, V.; Long, C. J.; Makwana, A.; Newbould, R. D.; Rabiner, E. A.; Ghatei, M. A.; Bloom, S. R.; Matthews, P. M.; Beaver, J. D.; Dhillo, W. S. The Gut Hormones PYY 3-36 and GLP-1 7-36 Amide Reduce Food Intake and Modulate Brain Activity in Appetite Centers in Humans. Cell Metab. 2011. https://doi.org/10.1016/j.cmet.2011.09.010.
30. Habib, A. M.; Richards, P.; Rogers, G. J.; Reimann, F.; Gribble, F. M. Co-Localisation and Secretion of Glucagon-like Peptide 1 and Peptide Y Y from Primary Cultured Human L Cells. Diabetologia 2013. https://doi.org/10.1007/s00125-013-2887-z.
31. Lee, Y. S.; Jun, H. S. Anti-Diabetic Actions of Glucagon-like Peptide-1 on Pancreatic Beta-Cells. Metabolism: Clinical and Experimental. 2014. https://doi.org/10.1016/j.metabol.2013.09.010.
32. Zhang, Y.; Ding, Y.; Zhong, X.; Guo, Q.; Wang, H.; Gao, J.; Bai, T.; Ren, L.; Guo, Y.; Jiao, X.; Liu, Y. Geniposide Acutely Stimulates Insulin Secretion in Pancreatic β-Cells by Regulating GLP-1 Receptor/CAMP Signaling and Ion Channels. Mol. Cell. Endocrinol. 2016. https://doi.org/10.1016/j.mce.2016.04.020.
33. Ten Kulve, J. S.; Veltman, D. J.; Van Bloemendaal, L.; Groot, P. F. C.; Ruhe, H. G.; Barkhof, F.; Diamant, M.; Ijzerman, R. G. Endogenous GLP1 and GLP1 Analogue Alter CNS Responses to Palatable Food Consumption. J. Endocrinol. 2016. https://doi.org/10.1530/JOE-15-0461.
34. Tough, I R.; Forbes, S.; Tolhurst, R.; Ellis, M.; Herzog, H.; Bornstein, J. C.; Cox, H. M. Endogenous Peptide Y Y and Neuropeptide y Inhibit Colonic Ion Transport, Contractility and Transit Differentially via Y 1 and Y 2 Receptors. Br. J. Pharmacol. 2011. https://doi.org/10.1111/j.1476-5381.2011.01401.x.
35. Hung, C. C. C.; Pirie, F.; Luan, J.; Lank, E.; Motala, A.; Yeo, G. S. H.; Keogh, J. M.; Wareham, N. J.; O'Rahilly, S.; Faroogi, I. S. Studies of the Peptide Y Y and Neuropeptide Y2 Receptor Genes in Relation to Human Obesity and Obesity-Related Traits. Diabetes 2004. https://doi.org/10.2337/diabetes.53.9.2461.
36. Field, B. C. T.; Wren, A. M.; Peters, V.; Baynes, K. C. R.; Martin, N. M.; Patterson, M.; Alsaraf, S.; Amber, V.; Wynne, K.; Ghatei, M. A.; Bloom, S. R. PYY3-36 and Oxyntomodulin Can Be Additive in Their Effect on Food Intake in Overweight and Obese Humans. Diabetes 2010. https://doi.org/10.2337/db09-1859.
37. Steinert, R. E.; Feinle-Bisset, C.; Asarian, L.; Horowitz, M.; Beglinger, C.; Geary, N. Ghrelin, C C K, GLP-1, and PYY(3-36): Secretory Controls and Physiological Roles in Eating and Glycemia in Health, Obesity, and after RYGB. Physiol. Rev. 2017. https://doi.org/10.1152/physrev.00031.2014.
38. Guida, C.; Stephen, S. D.; Watson, M.; Dempster, N.; Larraufie, P.; Marjot, T.; Cargill, T.; Rickers, L.; Pavlides, M.; Tomlinson, J.; Cobbold, J. F. L.; Zhao, C. M.; Chen, D.; Gribble, F.; Reimann, F.; Gillies, R.; Sgromo, B.; Rorsman, P.; Ryan, J. D.; Ramracheya, R. D. PYY Plays a Key Role in the Resolution of Diabetes Following Bariatric Surgery in Humans. EBioMedicine 2019. https://doi.org/10.1016/j.ebiom.2018.12.040.
39. Behary, P.; Tharakan, G.; Alexiadou, K.; Johnson, N.; Wewer Albrechtsen, N. J.; Kenkre, J.; Cuenco, J.; Hope, D.; Anyiam, O.; Choudhury, S.; Alessimii, H.; Poddar, A.; Minnion, J.; Doyle, C.; Frost, G.; Le Roux, C.; Purkayastha, S.; Moorthy, K.; Dhillo, W.; Holst, J. J.; Ahmed, A. R.; Toby Prevost, A.; Bloom, S. R.; Tan, T. M. Combined GLP-1, Oxyntomodulin, and Peptide Y Y Improves Body Weight and Glycemia in Obesity and Prediabetes/Type 2 Diabetes: A Randomized, Single-Blinded, Placebo-Controlled Study. Diabetes Care 2019. https://doi.org/10.2337/dcl9-0449.
40. Habib, A. M.; Richards, P.; Rogers, G. J.; Reimann, F.; Gribble, F. M. Co-Localisation and Secretion of Glucagon-like Peptide 1 and Peptide Y Y from Primary Cultured Human L Cells. Diabetologia 2013. https://doi.org/10.1007/s00125-013-2887-z.
41. Everard, A., & Cani, P. D. (2014). Gut microbiota and GLP-1. Reviews in endocrine & metabolic disorders, 15(3), 189-196. https://doi.org/10.1007/s11154-014-9288-6
42. Lund, M. L., Sorrentino, G., Egerod, K. L., Kroone, C., Mortensen, B., Knop, F. K., Reimann, F., Gribble, F. M., Drucker, D. J., de Koning, E., Schoonjans, K., Backhed, F., Schwartz, T. W., & Petersen, N. (2020). L-Cell Differentiation Is Induced by Bile Acids Through GPBAR1 and Paracrine GLP-1 and Serotonin Signaling. Diabetes, 69(4), 614-623. https://doi.org/10.2337/dbl9-0764
43. Goldspink, D. A., Lu, V. B., Miedzybrodzka, E. L., Smith, C. A., Foreman, R. E., Billing, L. J., Kay, R. G., Reimann, F., & Gribble, F. M. (2020). Labeling and Characterization of Human GLP-1-Secreting L-cells in Primary Ileal Organoid Culture. Cell reports, 31(13), 107833. https://doi.org/10.1016/i.celrep.2020.107833
44. Tamura, K., Minami, K., Kudo, M., Iemoto, K., Takahashi, H., & Seino, S. (2015). Liraglutide improves pancreatic Beta cell mass and function in alloxan-induced diabetic mice. PloS one, 10(5), e0126003. https://doi.org/10.1371/journal.pone.0126003
45. Rozengurt, N.; Wu, S. V.; Chen, M. C.; Huang, C.; Sternini, C.; Rozengurt, E. Colocalization of the α-Subunit of Gustducin with PYY and GLP-1 in L Cells of Human Colon. Am. J. Physiol.—Gastrointest. Liver Physiol. 2006. https://doi.org/10.1152/aipgi.00074.2006
46. Lebrun, L. J., Lenaerts, K., Kiers, D., Pais de Barros, J. P., Le Guern, N., Plesnik, J., Thomas, C., Bourgeois, T., Dejong, C., Kox, M., Hundscheid, I., Khan, N. A., Mandard, S., Deckert, V., Pickkers, P., Drucker, D. J., Lagrost, L., & Grober, J. (2017). Enteroendocrine L Cells Sense LPS after Gut Barrier Injury to Enhance GLP-1 Secretion. Cell reports, 21(5), 1160-1168. https://doi.org/10.1016/j.celrep.2017.10.008
47. Petersen, N.; Reimann, F.; Bartfeld, S.; Farin, H. F.; Ringnalda, F. C.; Vries, R. G. J.; Van Den Brink, S.; Clevers, H.; Gribble, F. M.; De Koning, E. J. P. Generation of 1 Cells in Mouse and Human Small Intestine Organoids. Diabetes 2014. https://doi.org/10.2337/dbl3-0991.
48. Coppola, S., Avagliano, C., Calignano, A., & Berni Canani, R. (2021). The Protective Role of Butyrate against Obesity and Obesity-Related Diseases. Molecules (Basel, Switzerland), 26(3), 682. https://doi.org/10.3390/molecules26030682
49. Cani, P. D.; Hoste, S.; Guiot, Y.; Delzenne, N. M. Dietary Non-Digestible Carbohydrates Promote L-Cell Differentiation in the Proximal Colon of Rats. Br. J. Nutr. 2007. https://doi.org/10.1017/S0007114507691648.
50. Pais, R.; Gribble, F. M.; Reimann, F. Signalling Pathways Involved in the Detection of Peptones by Murine Small Intestinal Enteroendocrine L-Cells. Peptides 2016. https://doi.org/10.1016/j.peptides.2015.07.019

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of producing a live cell construct comprising enteroendocrine cells and subtypes of enteroendocrine cells, the method comprising:
culturing stem cells that are capable of differentiating into enteroendocrine (EEC) cells on an upper surface of a cell support structure, the cell support structure having both an upper surface and a lower surface, until at least a portion of the upper surface of the cell support structure is substantially covered by the stem cells; and
causing the stem cells to differentiate into EEC cells by maintaining a thin layer of fluid at the upper surface of the support structure,
wherein the stem cells generate a live cell construct comprising a substantially continuous cell monolayer comprising EEC cells and subtypes of EEC cells.

2. The method of claim 1, wherein the thin layer of fluid comprises a liquid, slurry, hydrogel, and/or semi-solid materials.

3. The method of any of the above claims, wherein the thin layer of fluid at the upper surface is maintained in a range of about 0.001 mm to about 10 mm, optionally about 0.001 mm to about 1 mm, above the luminal side of the cell monolayer.

4. The method of any of the above claims, wherein the thin layer of fluid at the upper surface is maintained by adding hormones and/or compounds to the medium composition to induce luminal fluid liquid secretion, optionally wherein the hormones and/or compounds comprise chemicals (e.g. ionomycin), hormones (e.g. vasoactive intestinal peptide, serotonin, gastrin, etc.), cytokines, metabolites, bacteria and/or bacteria components, optionally further adding hypertonic medium to the luminal side to stimulate fluid secretion towards the luminal side to create a thick mucus layer.

5. The method of any of the above claims, further comprising adding chemical compounds (e.g., diterpenoid (e.g., forskolin), Methylxanthines (e.g., IBMX)), prebiotics (e.g., oligofructose and inulin-type fructans), hormone (e.g., serotonin, arginine vasopressin, angiotensin II), receptor agonists (e.g., BIMU8, Liraglutide (Saxenda), AM1638 (e.g., FFAR1 agonist), AR231453 (e.g., GPR119 agonist), and a GPBAR1 agonist), cytokines (e.g., IL-10, IL-6, TNF-α), bacteria or food metabolites (e.g., acetate, propionate and butyrate), bacteria and/or bacteria components (e.g., LPS, Akkermansia muciniphila, Bifidobacterium spp., Lactobacillus spp.) to the live cell construct to increase the density of the EEC cells.

6. The method of any of the above claims, further comprising adding chemical compounds (e.g., diterpenoid (e.g., forskolin), Methylxanthines (e.g., IBMX)), prebiotics (e.g., oligofructose and inulin-type fructans), hormone (e.g., serotonin, arginine vasopressin, angiotensin II), receptor agonists (e.g., BIU8, Liraglutide (Saxenda), AM1638 (e.g., FFAR1 agonist), AR231453 (e.g., GPR119 agonist), and a GPBAR1 agonist), cytokines (e.g., IL-10, IL-6, TNF-α), bacteria or food metabolites (e.g., acetate, propionate and butyrate), bacteria and/or bacteria components (e.g., LPS, Akkermansia muciniphila, Bifidobacterium spp., Lactobacillus spp.) to the live cell construct to mimic differentiation stages of EEC cells to further increase a density of the EEC cells.

7. The method of any of the above claims, wherein the thin layer of fluid is maintained by a microfluidic flow setup.

8. The method of any of the above claims, wherein high barrier integrity is maintained.

9. The method of any of the above claims, wherein the EEC cells secrete serotonin.

10. The method of any of the above claims, wherein the EEC cells secrete glucagon-like peptide-1 (GLP-1).

11. The method of any of the above claims, wherein the EEC cells secrete peptide YY (PYY), or other intestinal hormones including cholecystokinin, motilin, neurotensin, leptin and/or secretin.

12. The method of any of the above claims, wherein the EEC cells comprise L-cells.

13. The method of claim 12, wherein a density of the L-cells is greater than about 50 cells/mm², optionally greater than about 100 cells/mm².

14. The method of any of the above claims, wherein the method is used for screening of drug, metabolite, foodstuff or compound-induced secretion of hormones from EEC cells.

15. The method of any of the above claims, wherein the method is used for screening of drug, metabolite, foodstuff or compounds that block secretion of hormones from EEC cells.

16. The method of any of the above claims, wherein the method is used for screening of drug, metabolite, foodstuff or compounds that potentiate secretion of hormones from EEC cells.

17. The method of any of the above claims, wherein the gastrointestinal epithelial cells are selected from the group consisting of mammalian, avian, reptilian, amphibian, and insect cells.

18. The method of any of the above claims, wherein the gastrointestinal epithelial cells are human gastrointestinal epithelial cells.

19. The method of any of the above claims, wherein the gastrointestinal epithelial cells are selected from the group consisting of colon, small intestine, stomach, esophagus, tongue, nasopharynx, oropharynx, laryngeopharynx, and pancreatic epithelial cells.

20. The method of any of the above claims, further comprising adding one or more compounds in an expansion medium to prevent early lineage-fate decision of stem cells during a proliferation stage, optionally wherein the formation of EEC cells can be further enhanced by addition of the one or more compounds in the expansion medium.

21. The method of claim 20, wherein the one or more compounds are selected from a Wnt signaling activator and/or a Wnt signaling enhancer, optionally wherein the signaling activator comprises CHIR99021, WAY316606, ABC99, IQ1, and/or arylpyrimidine, and the Wnt signaling enhancer comprises proteins of Wnt-3A and/or R-spondin.

22. A live cell construct produced by the method of any of the above claims, wherein the live cell construct comprises a substantially continuous cell monolayer comprising EEC cells and subtypes of EEC cells.

23. The live cell construct of claim 22, wherein the EEC cells secrete serotonin.

24. The live cell construct of any of claims 22-23, wherein the EEC cells secrete glucagon-like peptide-1 (GLP-1).

25. The live cell construct of any of claims 22-24, wherein the EEC cells secrete peptide YY (PYY), or other intestinal hormones including cholecystokinin, motilin, neurotensin, leptin and/or secretin.

26. The live cell construct of any of claims 22-25, wherein the EEC cells comprise L-cells.
wherein a density of the L-cells is greater than about 50 cells/mm², optionally greater than about 100 cells/mm².

27. A live cell culture system, the system comprising:
a cell support structure, the cell support structure having both an upper surface and a lower surface, the cell support structure further comprising a porous carrier on the upper surface,
a culture vessel housing the cell support structure and providing a contained area for a culture medium, wherein the culture medium is contained below the lower surface of the cell support structure,
wherein the cell support structure is configured to generate a live cell construct from stem cells seeded on the cell support structure, the system configured to cause the stem cells to differentiate into enteroendocrine (EEC) cells by maintaining a thin layer of fluid at the upper surface of the support structure, wherein the thin layer of fluid at the upper surface is maintained by hormones and/or compounds in the culture medium contained in the culture vessel inducing luminal fluid liquid secretion.

28. The system of claim 27, wherein the system is configured to generate a substantially continuous cell monolayer comprising EEC cells and subtypes of EEC cells.

29. The system of any of claims 27-28, wherein the thin layer of fluid is maintained by a microfluidic flow of the cell support structure.

30. The system of any of claims 27-29, wherein the culture vessel comprises a multi-well plate, culture dish, vial or tube.

31. The system of any of claims 27-30, wherein the thin layer of fluid at the upper surface is maintained by hormones and/or compounds in the culture medium that induces luminal fluid liquid secretion, optionally wherein the hormones and/or compounds comprise chemicals (e.g. ionomycin), hormones (e.g. vasoactive intestinal peptide, serotonin, gastrin, etc.), cytokines, metabolites, bacteria and/or bacteria components, optionally further adding hypertonic medium to the luminal side to stimulate fluid secretion towards the luminal side to create a thick mucus layer.

32. A method of screening a test compound or microbe for a toxicological, physiological, or carcinogenic effect, comprising:
(a) providing a cell construct according to any of the above claims;
(b) contacting a test compound or microbe to the cell construct; and then
(c) detecting a toxicological, pharmacologic physiological, or carcinogenic effect of the microbe on cells of the cell construct, optionally by comparing the cell construct after the contacting to a like cell construct to which the compound or microbe has not been contacted, and/or by comparing the cell construct after contacting with the cell construct before the contacting step.

33. The method of claim 32, wherein the test compound or microbe is selected from the group consisting of aromatic organic compounds, aliphatic organic compounds, and mixed aromatic and aliphatic organic compounds.

34. The method of any of claims 32-33, wherein the test compound or microbe is selected from the group consisting of gram negative bacteria, gram positive bacteria, yeast, and molds.

35. The method of any of claims 32-34, comprising screening for pharmacologic interventions for diabetes.

36. The method of any of claims 32-35, comprising screening for pharmacologic interventions for obesity.
